(19) United States
(12) Patent Application Publication
(10) Pub. No.: US 2021/0139902 A1
(43) Pub. Date: May 13, 2021

(54) RNAI TARGET GENE THAT IS HIGHLY LETHAL TO APHIDS AND USE THEREOF

(71) Applicant: CAS Center For Excellence in Molecular Plant Sciences, Shanghai (CN)

(72) Inventors: Xuexia Miao, Shanghai (CN); Haichao Li, Shanghai (CN); Ruobing Guan, Shanghai (CN)

(73) Assignee: CAS Center For Excellence in Molecular Plant Sciences, Shanghai (CN)

(21) Appl. No.: 17/097,574

(22) Filed: Nov. 13, 2020

Related U.S. Application Data
(63) Continuation of application No. PCT/CN2019/086896, filed on May 14, 2019.

(30) Foreign Application Priority Data
May 14, 2018 (CN) 201810455499.3

(51) Int. Cl. C12N 15/113; A01N 63/60; C12N 15/82

(52) U.S. Cl. CPC C12N 15/113; C12N 2310/14; C12N 15/8218; A01N 63/60

(57) ABSTRACT

Provided are a RNAi target gene that is lethal to aphids and the use thereof. Specifically, provided are six gene fragments resulting in the death of aphid nymphs and/or death thereof in the adult stage based on RNA interference technology. The death of the aphids can be caused by spraying a dsRNA-containing composition onto plants to feed aphids or directly spraying same onto the skin of the aphids.

### TECHNICAL FIELD

The present invention belongs to the fields of biotechnology and agricultural applications. Specifically, the present invention relates to RNAi target genes that are highly effective in killing aphids and uses thereof.

### BACKGROUND

Aphid is an important worldwide pest. It belongs to Hemiptera, Aphidoidea. At present, more than 4,700 kinds of aphids are known. They are small in size and fast in reproduction. They are important agricultural and horticultural pests. For the prevention and control of aphids, currently it is still dominated by chemical agents. However, due to its fast reproduction speed and strong concealment, its control effect is poor, and a large amount of pesticides are required to inhibit its reproduction, which inevitably leads to resistance of aphids.

RNAi is widely used as a tool for gene function research, especially in animals and plants with imperfect genetic manipulation tools. However, currently in insects, after dsRNA enters the insect body through feeding, it must enter the cell to activate the RNAi mechanism. Insect intestinal wall cells can prevent most dsRNA from entering other tissues, which is a key factor affecting the efficiency of RNAi, and is also the biggest obstacle in the application of dsRNA oral delivery methods.

Because different kinds of insects have different dsRNA uptake mechanisms, leading to differences in their response to dsRNA and target gene silencing efficiency. Therefore, the lethal effects of different kinds of insects are quite different.

Therefore, there is an urgent need in the art to develop an RNAi target gene that is highly effective in killing aphids.

### SUMMARY OF THE INVENTION

The purpose of the present invention is to provide an RNAi target gene that is highly effective in killing aphids.

In a first aspect of the present invention, it provides a dsRNA construct, the dsRNA construct is double-stranded, and its positive or negative strand contains a structure as shown in Formula I:

Seq_{forward}-X-Seqᵣₑᵥₑᵣₛₑ Formula I

wherein

Seq_{forward} is a nucleotide sequence of insect nymph and/or adult stage regulation-related gene or fragment;

Seqᵣₑᵥₑᵣₛₑ is a nucleotide sequence that is basically complementary to Seq_{forward};

X is an intervening sequence between the Seq_{forward} and the Seqᵣₑᵥₑᵣₛₑ, and the intervening sequence is not complementary to the Seq_{forward} and the Seqᵣₑᵥₑᵣₛₑ,

wherein the insect nymph and/or adult stage regulation-related gene is selected from the group consisting of DS7 gene, DS9 gene, DS15 gene, DS25 gene, DS27 gene, DS45 gene and a combination thereof.

In another preferred embodiment, the length of the dsRNA is at least 21nt.

In another preferred embodiment, for the DS7 gene, the length of the dsRNA is 21nt-1350nt, preferably 506nt-1093nt.

In another preferred embodiment, for the DS9 gene, the length of the dsRNA is 21nt-909nt, preferably 54nt-631nt.

In another preferred embodiment, for the DS15 gene, the length of the dsRNA is 21nt-2148nt, preferably 516nt-1029nt.

In another preferred embodiment, for the DS25 gene, the length of the dsRNA is 21nt-1233nt, preferably 58nt-674nt.

In another preferred embodiment, for the DS27 gene, the length of the dsRNA is 21nt-1152nt, preferably, 219nt-748nt.

In another preferred embodiment, for the DS45 gene, the length of the dsRNA is 21nt-909nt, preferably 42nt-637nt.

In another preferred embodiment, the homology with the dsRNA is at least 80%, preferably, 85%-100%.

In another preferred embodiment, the length of the Seq_{forward} and the Seqᵣₑᵥₑᵣₛₑ is at least 50 bp.

In another preferred embodiment, the dsRNA construct can form a dsRNA as shown in Formula II,

[chemical structure]

wherein

Seq′_{forward} is a RNA sequence or sequence fragment corresponding to the Seq_{forward} sequence;

Seq′ᵣₑᵥₑᵣₛₑ is a sequence that is basically complementary to the Seq′_{forward};

X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ,

∥ represents the hydrogen bond formed between Seq_{forward} and Seqᵣₑᵥₑᵣₛₑ.

In another preferred embodiment, the dsRNA is dsRNA without loop.

In another preferred embodiment, the dsRNA is amplified from the sequence as shown in SEQ ID NO. 9-10.

In another preferred embodiment, the dsRNA is amplified from the sequence as shown in SEQ ID NO. 11-12.

In another preferred embodiment, the dsRNA is amplified from the sequence as shown in SEQ ID NO. 13-14.

In another preferred embodiment, the dsRNA is amplified from the sequence as shown in SEQ ID NO. 15-16.

In another preferred embodiment, the dsRNA is amplified from the sequence as shown in SEQ ID NO. 17-18.

In another preferred embodiment, the dsRNA is amplified from the sequence as shown in SEQ ID NO. 19-20.

In a second aspect of the present invention, it provides a dsRNA as shown in Formula II,

[chemical structure]

wherein

Seq′_{forward} is a RNA sequence or sequence fragment corresponding to a nucleotide sequence of an insect nymph and/or adult stage regulation-related gene or fragment;

Seq′ᵣₑᵥₑᵣₛₑ is a sequence that is basically complementary to the Seq′_{forward}; X′ is none; or is an intervening sequence between Seq′_{forward} and Seq′ᵣₑᵥₑᵣₛₑ; and the intervening sequence is not complementary to Seq′_{forward} and Seq′ᵣₑᵥₑᵣₛₑ;

wherein, the insect nymph and/or adult stage regulation-related gene is selected from the group consisting of DS7 gene, DS9 gene, DS15 gene, DS25 gene, DS27 gene, DS45 gene, and a combination thereof;

∥ represents the hydrogen bond formed between Seq_{forward} and Seqᵣₑᵥₑᵣₛₑ.

In another preferred embodiment, the length of the Seq_{forward} and Seqᵣₑᵥₑᵣₛₑ is at least 50 bp.

In another preferred embodiment, the length of the intervening sequence X′ is 0-300 bp.

In another preferred embodiment, the nymph and/or adult stage regulation-related gene is derived from the Aphis.

In another preferred embodiment, the sequence of the DS7 gene is shown in SEQ ID NO. 1 or 24.

In another preferred embodiment, the sequence of the DS9 gene is shown in SEQ ID NO. 2 or 25.

In another preferred embodiment, the sequence of the DS15 gene is shown in SEQ ID NO. 3 or 26.

In another preferred embodiment, the sequence of the DS25 gene is shown in SEQ ID NO. 4 or 27.

In another preferred embodiment, the sequence of the DS27 gene is shown in SEQ ID NO. 5 or 28.

In another preferred embodiment, the sequence of the DS45 gene is shown in SEQ ID NO. 6 or 29.

In another preferred embodiment, the insect is a phytophagous insect, preferably a homoptera insect, most preferably Aphis.

In another preferred embodiment, the insect is selected from the group consisting of green peach aphid, soybean aphid, and a combination thereof.

In a third aspect of the present invention, it provides an expression vector containing the dsRNA construct according to the first aspect of the present invention.

In a fourth aspect of the present invention, it provides a host cell that contains the expression vector according to the third aspect of the present invention or the DNA sequence corresponding to the dsRNA construct according to the first aspect of the present invention is integrated into the chromosome.

In another preferred embodiment, the host cell is a plant cell, preferably a green leaf plant cell.

In another preferred embodiment, the plant includes a cruciferous plant (such as a vegetable or soybean).

In a fifth aspect of the present invention, it provides a composition comprising the dsRNA construct according to the first aspect of the present invention and/or the dsRNA according to the second aspect of the present invention, and an acceptable carrier for insect feeding.

In another preferred embodiment, the acceptable carrier for insect feeding includes water.

In another preferred embodiment, the composition is a composition used to induce or cause the death of aphis nymphs and/or adult stage.

In another preferred embodiment, the dsRNA has the following sequence:

dsRNA1: having a sequence corresponding to SEQ ID NO. 1 or 24;

dsRNA2: having a sequence corresponding to SEQ ID NO. 2 or 25;

dsRNA3: having a sequence corresponding to SEQ ID NO. 3 or 26;

dsRNA4: having a sequence corresponding to SEQ ID NO. 4 or 27;

dsRNA5: having a sequence corresponding to SEQ ID NO. 5 or 28;

dsRNA6: having a sequence corresponding to SEQ ID NO. 6 or 29.

In another preferred embodiment, the DS7 gene, DS9 gene, DS15 gene, DS25 gene, DS27 gene, and/or DS45 gene is from an insect, preferably from a Homoptera insect, and most preferably from Aphis.

In another preferred embodiment, the content of dsRNA1 in the pharmaceutical composition is 1-500 ng/μl, preferably 5-300 ng/μl, more preferably 50-150 ng/μl.

In another preferred embodiment, the content of dsRNA2 in the pharmaceutical composition is 1-500 ng/μl, preferably 5-300 ng/μl, more preferably 50-150 ng/μl.

In another preferred embodiment, the content of dsRNA3 in the pharmaceutical composition is 1-500 ng/μl, preferably 5-300 ng/μl, more preferably 50-150 ng/μl.

In another preferred embodiment, the content of dsRNA4 in the pharmaceutical composition is 1-500 ng/μl, preferably 5-300 ng/μl, more preferably 50-150 ng/μl.

In another preferred embodiment, the content of dsRNA5 in the pharmaceutical composition is 1-500 ng/μl, preferably 5-300 ng/μl, more preferably 50-150 ng/μl.

In another preferred embodiment, the content of dsRNA6 in the pharmaceutical composition is 1-500 ng/μl, preferably 5-300 ng/μl, more preferably 50-150 ng/μl.

In a sixth aspect of the present invention, it provides a use of the dsRNA construct according to the first aspect of the present invention, or the dsRNA according to the second aspect of the present invention, or the host cell according to the fourth aspect of the present invention, or the composition according to the fifth aspect of the present invention, which is selected from the group consisting of:

(1) improving the control effect of aphids; and/or

(2) increasing the dropping rate of insect population; and/or

(3) decreasing the expression level of nymph and/or adult stage regulation-related gene; and/or

(4) reducing the initial number of insect population; and/or

(5) reducing plant damage rate; and/or

(6) reducing crop damage degree and improving the quality of crop products.

In a seventh aspect of the present invention, it provides a method for killing insects, comprising the steps of: using an interference molecule that interferes with the expression of an insect nymph and/or adult stage regulation-related gene, or feeding or spraying an insect with a vector, cell, plant tissue or insect prevention and control reagent containing the interference molecule;

preferably, the insect nymph and/or adult stage regulation-related gene is selected from the group consisting of DS7 gene, DS9 gene, DS15 gene, DS25 gene, DS27 gene, DS45 gene, and a combination thereof.

In another preferred embodiment, the killing insects includes:

(1) improving the control effect of aphids; and/or

(2) increasing the dropping rate of insect population; and/or

(3) decreasing the expression level of nymph and/or adult stage regulation-related gene; and/or

(4) reducing the initial number of insect population; and/or

(5) reducing plant damage rate; and/or

(6) reducing crop damage degree and improving the quality of crop products.

In another preferred embodiment, the interference molecule is selected from: dsRNA, antisense nucleic acid, small interfering RNA, and microRNA that use an insect nymph and/or adult stage regulation-related gene or a fragment thereof or a transcript thereof as a target for inhibiting or silencing.

In another preferred embodiment, the insect nymph and/or adult stage regulation-related gene is derived from the Aphis.

In another preferred embodiment, the insect is a phytophagous insect, preferably from a Hemiptera insect, and most preferably from the Aphis.

In another preferred embodiment, the method includes the steps of: using the dsRNA construct according to the first aspect of the present invention, or the dsRNA according to the second aspect of the present invention, or the host cell according to the fourth aspect of the present invention, or the composition according to the fifth aspect of the present invention to feed or spray insects.

In an eighth aspect of the present invention, it provides a method for preparing the dsRNA according to the second aspect of the present invention, comprising the steps:

(i) preparing a construct expressing dsRNA, and the construct is double-stranded, and its positive or negative strand contains a structure as shown in Formula I:

Seq_{forward}-X-Seqᵣₑᵥₑᵣₛₑ Formula I

wherein

Seq_{forward} is a nucleotide sequence of insect nymph and/or adult stage regulation-related gene or fragment;

Seqᵣₑᵥₑᵣₛₑ is a nucleotide sequence that is basically complementary to Seq_{forward};

X is an intervening sequence located between the Seq_{forward} and the Seqᵣₑᵥₑᵣₛₑ, and the intervening sequence is not complementary to the Seq_{forward} and the Seqᵣₑᵥₑᵣₛₑ,

wherein the insect nymph and/or adult stage regulation-related gene is selected from the group consisting of DS7 gene, DS9 gene, DS15 gene, DS25 gene, DS27 gene, DS45 gene and a combination thereof;

(ii) transforming the construct as described in step (i) into a host cell, thereby expressing and forming a dsRNA as shown in Formula II in the host cell,

[chemical structure]

wherein

Seq′_{forward} is a RNA sequence or sequence fragment corresponding to the Seq_{forward} sequence;

Seq′ᵣₑᵥₑᵣₛₑ is a sequence that is basically complementary to the Seq′_{forward};

X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ,

∥ represents the hydrogen bond formed between Seq_{forward} and Seqᵣₑᵥₑᵣₛₑ.

In a ninth aspect of the present invention, it provides a method for preparing an insect prevention and control reagent comprising the steps of: spraying the dsRNA construct according to the first aspect of the present invention, or the dsRNA according to the second aspect of the present invention, or the host cell according to the fourth aspect of the present invention, or the composition according to the fifth aspect of the present invention on the surface of the plant, thereby producing the insect prevention and control agent.

In another preferred embodiment, the plant is selected from the group consisting of soybean, radish, peach tree, tobacco, and a combination thereof.

In a tenth aspect of the present invention, it provides a method for improving a plant resistance to an insect, comprising:

expressing a recombinant DNA construct in a plant, wherein the recombinant DNA construct comprises DNA encoding RNA, and the RNA has a sequence that is substantially identical or substantially complementary to at least 21 or more consecutive nucleotides of the target gene, wherein the target gene is an insect nymph and/or adult stage regulation-related gene, selected from the group consisting of DS7 gene, DS9 gene, DS15 gene, DS25 gene, DS27 gene, DS45 gene, and a combination thereof.

In another preferred embodiment, the target gene is selected from the group consisting of:

(i) a polynucleotide whose sequence is shown in any one of SEQ ID NO. 1-6 and 24-29;

(ii) a polynucleotide whose nucleotide sequence is ≥80%, preferably 85%-90%, more preferably, 95%, 96%, 97%, 98%, 99% or 100% homologous to the sequence as shown in any one of SEQ ID NO. 1-6 and 24-29;

(iii) a polynucleotide in which 1-60 (preferably 1-30, more preferably 1-10) nucleotides are truncated or added to the 5′end and/or 3′end of the polynucleotide as shown in any one of SEQ ID NO. 1-6 and 24-29;

(iv) a polynucleotide which is complementary to any one of the polynucleotides as described in (i) to (iii).

In another preferred embodiment, the target gene is as shown in any one of SEQ ID NO. 1-6 and 24-29.

In another preferred embodiment, the homology with the RNA is at least 80%, preferably, 85%-100%, more preferably, 95-100%.

In another preferred embodiment, for the DS7 gene, the RNA has a sequence that is substantially identical or substantially complementary to the 21nt-1350nt, preferably 506nt-1093nt consecutive nucleotides of the target gene.

In another preferred embodiment, for the DS9 gene, the RNA has a sequence that is substantially identical or substantially complementary to the 21nt-909nt, preferably 54nt-631nt consecutive nucleotides of the target gene.

In another preferred embodiment, for the DS15 gene, the RNA has a sequence that is substantially identical or substantially complementary to the 21nt-2148nt, preferably 516nt-1029nt consecutive nucleotides of the target gene.

In another preferred example, for the DS25 gene, the RNA has a sequence that is substantially identical or substantially complementary to the 21nt-1233nt, preferably 58nt-674nt consecutive nucleotides of the target gene.

In another preferred example, for the DS27 gene, the RNA has a sequence that is substantially identical or substantially complementary to the 21nt-1152nt, preferably 219nt-748nt consecutive nucleotides of the target gene.

In another preferred example, for the DS45 gene, the RNA has a sequence that is substantially identical or substantially complementary to the 21nt-909nt, preferably 42nt-637nt consecutive nucleotides of the target gene.

In another preferred embodiment, the RNA is a dsRNA containing at least one RNA strand.

In another preferred embodiment, the RNA strand includes a sequence having at least 90%, preferably 95-100% homology with any one of a sequence selected from the group consisting of SEQ ID NO. 1-6 and 24-29.

In another preferred embodiment, the recombinant DNA construct contains a promoter, preferably a heterologous promoter.

In another preferred embodiment, the promoter is selected from the group consisting of a constitutive promoter, a space-specific promoter, a time-specific promoter, a development-specific promoter, an inducible promoter, or a combination thereof.

In another preferred embodiment, the promoter is a promoter that is functional in a plant.

In another preferred embodiment, the promoter is selected from the group consisting of pol II promoter, pol III promoter, pol IV promoter, pol V promoter, and a combination thereof.

In another preferred embodiment, the recombinant DNA construct further comprises one or more other elements selected from the group consisting of enhancers, small RNA recognition sites, aptamers or ribozymes, terminators, and additional and extra expression cassettes for expressing coding sequences (for example, expressing transgenes, such as insecticidal proteins or selectable markers), non-coding sequences (for example, expressing additional inhibitory elements), and a combination thereof.

In another preferred embodiment, the plant further expresses one or more insecticidal proteins selected from the group consisting of patatin, phytolectin, plant steroid, Bacillus thuringiensis insecticidal protein, Xenorhabdus insecticidal protein, Photorhabdus insecticidal protein, Bacillus late blight insecticidal protein, and Bacillus sphaericus insecticidal protein.

In another preferred embodiment, the plant includes a angiosperm and a gymnosperm.

In another preferred embodiment, the gymnosperm is selected from the group consisting of Cycadaceae, Podocarpaceae, Araucariaceae, Pinaceae, Taxodiaceae, Cupressaceae, Cephalotaxaceae, Taxaceae, Ephedraceae, Gnetaceae, monotypic family, Welwitschiaceae, and a combination thereof.

In another preferred embodiment, the plant includes monocotyledonous plants and dicotyledonous plants.

In another preferred embodiment, the plant includes a herbaceous plant and a woody plant.

In another preferred embodiment, the herbaceous plant is selected from the group consisting of Solanaceae, a gramineous plant, a leguminous plant, and a combination thereof.

In another preferred embodiment, the woody plant is selected from the group consisting of Actinidiaceae, Rosaceae, Moraceae, and a combination thereof.

In another preferred embodiment, the plant is selected from the group consisting of a cruciferous plant, a gramineous plant, a leguminous plant, Solanaceae, Actinidiaceae, Malvaceae, Paeoniaceae, Rosaceae, Liliaceae, and a combination thereof.

In another preferred embodiment, the plant is selected from the group consisting of Arabidopsis thaliana, Oryza sativa, Chinese cabbage, soybean, tomato, corn, tobacco, wheat, sorghum, radish, and a combination thereof.

In an eleventh aspect of the present invention, it provides a method for preparing a transgenic plant cell, comprising the steps:

(i) introducing or transfecting a recombinant DNA construct into a plant cell so that the plant cell contains the construct, thereby producing the transgenic plant cell, wherein the recombinant DNA construct contains DNA encoding RNA, the RNA has a sequence that is substantially identical or substantially complementary to at least 21 or more consecutive nucleotides of the target gene, wherein the target gene is an insect nymph and/or adult stage regulation-related gene, selected from the group consisting of DS7 Gene, DS9 gene, DS15 gene, DS25 gene, DS27 gene, DS45 gene, and a combination thereof.

In another preferred embodiment, the target gene is selected from the group consisting of:

(i) a polynucleotide whose sequence is shown in any one of SEQ ID NO. 1-6 and 24-29;

(ii) a polynucleotide whose nucleotide sequence is ≥80%, preferably 85%-90%, more preferably, 95%, 96%, 97%, 98%, 99% or 100% homologous to the sequence as shown in any one of SEQ ID NO. 1-6 and 24-29; (Please review)

(iii) a polynucleotide in which 1-60 (preferably 1-30, more preferably 1-10) nucleotides are truncated or added to the 5′end and/or 3′end of the polynucleotide as shown in any one of SEQ ID NO. 1-6 and 24-29;

(iv) a polynucleotide which is complementary to any of the polynucleotides as described in (i) to (iii).

In another preferred embodiment, the homology with the RNA is at least 80%, preferably, 85%-100%, more preferably, 95-100%.

In another preferred embodiment, for the DS7 gene, the RNA has a sequence that is substantially identical or substantially complementary to the 21nt-1350nt, preferably 506nt-1093nt consecutive nucleotides of the target gene.

In another preferred embodiment, for the DS9 gene, the RNA has a sequence that is substantially identical or substantially complementary to the 21nt-909nt, preferably 54nt-631nt consecutive nucleotides of the target gene.

In another preferred embodiment, for the DS15 gene, the RNA has a sequence that is substantially identical or substantially complementary to the 21nt-2148nt, preferably 516nt-1029nt consecutive nucleotides of the target gene.

In another preferred example, for the DS25 gene, the RNA has a sequence that is substantially identical or substantially complementary to the 21nt-1233nt, preferably 58nt-674nt consecutive nucleotides of the target gene.

In another preferred example, for the DS27 gene, the RNA has a sequence that is substantially identical or substantially complementary to the 21nt-1152nt, preferably 219nt-748nt consecutive nucleotides of the target gene.

In another preferred example, for the DS45 gene, the RNA has a sequence that is substantially identical or substantially complementary to the 21nt-909nt, preferably 42nt-637nt consecutive nucleotides of the target gene.

In another preferred embodiment, the transfection adopts the Agrobacterium transformation method or the gene gun bombardment method.

In a twelfth aspect of the present invention, it provides a method for preparing a transgenic plant, comprising the steps:

regenerating a transgenic plant cell prepared by the method according to the eleventh aspect of the present invention into a plant body, thereby obtaining the transgenic plant.

In a thirteenth aspect of the present invention, it provides a transgenic plant cell prepared by the method according to the eleventh aspect of the present invention.

In a fourteenth aspect of the present invention, it provides a transgenic plant prepared by the method according to the twelfth aspect of the present invention.

It should be understood that, within the scope of the present invention, the technical features specifically described above and below (such as the Examples) can be combined with each other, thereby constituting a new or preferred technical solution which needs not be described one by one limited to the length.

### DESCRIPTION OF DRAWINGS

FIG. 1 shows the control effect of target genes on aphids.

FIG. 2 shows the detection results of the relative expression levels of target genes.

FIG. 3 shows the control effect of the three target genes of Myzus persicae in the field.

FIG. 4 shows the results of statistical analysis of the field control effect and the dropping rate of insect of Myzus persicae.

### DETAILED DESCRIPTION OF INVENTION

After extensive and intensive research, the inventors have screened the aphid nymph and/or adult stage regulation-related gene fragments, and unexpectedly found that for the DS7 gene as shown in SEQ ID NO. 1 or 24, the DS9 gene as shown in SEQ ID NO. 2 or 25, the DS15 gene as shown in SEQ ID NO. 3 or 26, DS25 gene as shown in SEQ ID NO. 4 or 27, DS27 gene as shown in SEQ ID NO. 5 or 28, DS45 gene as shown in SEQ ID NO. 6 or 29, interfering RNA (dsRNA) is synthesized, and the dsRNA is fed by phytophagous insects (such as Aphis) or directly sprayed on the surface of the phytophagous insects, thereby interfering with target genes, inhibiting the expression of target genes, and finally killing aphids. The present invention can also construct plants that can improve insect resistance, and the method of the present invention can also effectively kill aphids, the control effect of aphids is ≥80%, and the dropping rate of insect is ≥70%. On this basis, the present inventor has completed the present invention.

Terms

As used herein, the “crop” refers to various plants cultivated in agriculture, including food crops, economic crop (oil crops, vegetable crops, flowers, grasses, trees), industrial crops, feed crops, herb crops, etc., and can grow into large quantities or harvest large areas for profit or provisions (such as cereal, vegetables, cotton, flax, etc.).

Among them, food crops are mainly rice, corn, beans, potatoes, highland barley, broad beans and wheat; oil crops are mainly oilseeds, vines, big mustard, peanuts, flax, hemp, sunflower, etc.; Vegetable crops mainly include radishes, Chinese cabbage, celery, leeks, garlic, Green onions, carrots, Cucumis melo var flexuosus, lotus vegetables, Jerusalem artichokes, sword bean, coriander, asparagus lettuce, citron day-lily, peppers, cucumbers, tomatoes, coriander, etc.; fruits include pears, green plums, apples, peaches, Apricots, walnuts, plums, cherries, strawberries, crabapple, red dates and other varieties; wild fruits include Pyrus ussuriensis, Armeniaca vulgaris Lam, wild peach, Ziziphus jujuba var. spinosa, prunus maackii, sea-buckthorn, etc.; feed crops are such as corn, green manure, Astragalus sinicus, etc.; medicinal crops are ginseng, Angelica sinensis, Lonicera japonica, mint, mugwort, etc.

RNA Interference (RNAi)

As used herein, the term “RNA interfering (RNAi)” refers to: some small double-stranded RNA can efficiently and specifically block the expression of specific genes in vivo, promote mRNA degradation, and induce cells to show a phenotype with a specific gene deletion, which is also called RNA intervention or RNA interference. RNA interference is a highly specific gene silencing mechanism at the mRNA level.

As used herein, the term “small interfering RNA (siRNA)” refers to a short double-stranded RNA molecule that can target mRNA with homologous complementary sequences to degrade specific mRNA. This process is the RNA interference pathway.

In the present invention, the basic principle of RNA interference is: using plants as a medium to make insects eat small interfering RNA (siRNA) that can interfere with their gene (such as DS7 genes, DS9 genes, DS15 genes, DS25 genes, DS27 genes, DS45 genes) expression, thereby inhibiting the growth of insects.

Specifically, the principle is: through aphids' herbivorous feeding or spraying interfering substances on aphids, RNAi enters the insect body and interferes with the RNA of the target gene and inhibits the expression of the target gene, thereby interfering with the normal growth and development of the insect, causing the death of aphids.

As a preferred way, an intron sequence is used to connect complementary gene sequences at both ends. After being introduced into the cell, a “neck-loop” structure can be produced, and the “neck”-shaped part can be processed into small RNAs of about 21-25nt in the insect body, which can effectively inhibit the expression of target genes.

As another preferred way, using the T7 primers in Table 1 to amplify respectively, the double-stranded RNA is formed by complementary transcription, and this double-stranded RNA can be directly used to inhibit the expression of the target gene.

Insect Gene

As used herein, the term “insect gene” refers to a gene related to insect nymph and/or adult stage regulation. In a preferred embodiment of the present invention, the insect gene is DS7 gene, DS9 gene, DS15 gene, DS25 gene, DS27 gene, and/or DS45 gene. Low or non-expression of the gene will cause abnormalities in the growth, development, metabolism, reproduction and other processes of the insects, and even lead to the death of the insects.

As a preferred mode of the present invention, the length of the preferred insect gene fragment of the present invention is at least 21 bp, such as may be 30 bp, 50 bp, 60 bp, 80 bp, 100 bp, 200 bp, 500 bp, 1000 bp or the full length of the gene. When the gene is used in the present invention, it can be a full-length gene or a gene fragment. Preferably, the fragment for the DS7 gene is shown in SEQ ID NO: 24, the fragment for the DS9 gene is shown in SEQ ID NO: 25, the fragment for the DS15 gene is shown in SEQ ID NO. 26, the fragment for the DS25 gene is shown in SEQ ID NO. 27, the fragment for the DS27 gene is shown in SEQ ID NO. 28, the fragment for the DS45 gene is shown in SEQ ID NO. 29. The similarities between these fragments and these genes are 85%-100%, respectively, which can produce the same insecticidal effect.

The present invention also provides dsRNA for the DS50 gene, the sequence of the DS50 gene is shown in SEQ ID NO:23. Compared with DS7 gene, DS9 gene, DS15 gene, DS25 gene, DS27 gene, and/or DS45 gene, the control effect of DS50 gene is not good, the maximum is only about 23%.

The present invention provides interfering RNA targeting insect nymph and/or adult stage regulation-related genes. Insects can take up the interfering RNAi by oral administration of plants sprayed with RNAi or expressing dsRNA constructs or dsRNA, or spraying interfering RNAi directly on the surface of insects.

The dsRNA construct shown in the present invention is shown in Formula I, and the dsRNA is shown in Formula II. The length of the intervening sequence X used is not particularly limited, as long as it forms a construct with the forward sequence and the reverse sequence and when introduced into the body, it can form a dsRNA represented by Formula II. As a preferred mode of the present invention, the length of the intervening sequence of the present invention is 80-300 bp; more preferably 100-250 bp.

In a preferred embodiment of the present invention, the construct for expressing insect gene dsRNA is introduced into a host cell. The host cell can be a plant cell, tissue or organ, and the construct can express an insect gene dsRNA in a plant, and dsRNA is processed into siRNA. Generally, the length of siRNA is about 21-25 nt.

Usually, the construct is located on an expression vector. The expression vector usually also contains a promoter, an origin of replication, and/or a marker gene, etc. Methods well known to those skilled in the art can be used to construct the expression vector required by the present invention. These methods include in vitro recombinant DNA technology, DNA synthesis technology, and in vivo recombination technology, etc. The expression vector preferably contains one or more selectable marker genes to provide phenotypic traits for selection of transformed host cells, such as kamamycin, gentamicin, hygromycin, and ampicillin resistance.

A vector containing the above-mentioned appropriate gene sequence and appropriate promoter or control sequence can be used to transform an appropriate host. In the method of the present invention, the host may be any host suitable for carrying the expression vector and capable of delivering the expression vector to plant cells. Preferably, the host is Agrobacterium.

Although the insects exemplified in the examples of the present invention are aphids. However, it should be understood that the present invention has no particular limitations on the insects applicable to the present invention. The insects may be any phytophagous insects that can feed on plants, for example, they may be Hemiptera insects.

The present invention has no particular limitations on the plants applicable to the present invention. Plants eaten by aphids are preferred, such as soybeans, radishes, peach trees, tobacco and the like.

DS7 Gene

As used herein, the terms “DS7 gene”, “tubulin alpha chain-like”, and “tubulin a chain” can be used interchangeably, and they are all widely distributed globular proteins and are the basic structural unit of microtubules in cells. It plays an important role in cell movement and division, and is expressed in the nymph stage.

In the present invention, some glutamic acid residues at the C-terminus of the protein are polyglutamylated, resulting in a polyglutamic acid chain on the γ-carboxyl group. Polyglutamylation plays a key role in spastin (SPAST) microtubule cutting. SPAST preferentially recognizes and acts on microtubules modified with short polyglutamic acid tails: the cleavage activity of SPAST increases as the number of glutamate per tubulin increasing from 1 to 8, but the decrease exceeds the glutamylation threshold.

Some glutamic acid residues at the C-terminus are monoglycosylated, but not polyglycerinated. Monoglycination is mainly limited to tubulin (cilia and flagella) incorporated into axoneme. Both polypentanoylation and monoglycination can coexist on the same protein on adjacent residues, and reducing the level of glycylation can increase polypentanoylation and interact with each other.

In one embodiment of the present invention, based on RNAi technology, the DS7 gene is used as a target to screen interfering RNA fragments against the DS7 gene. Preferably, the sequence of the DS7 gene fragment is shown in SEQ ID NO:1 or 24:

(SEQ ID NO.: 1)
ATGCGTGAATGTATCTCTGTACACGTTGGCCAAGCTGGTGTTCAAAT
CGGTAATGCCTGCTGGGAATTGTACTGTTTGGAACATGGAATTGCTCCAG
ATGGTCAAATGCCATCTGACAAGACCATTGGAGGTGGAGACGACAGCTTC
AACACCTTCTTCAGCGAAACTGGCTCAGGCAAACATGTGCCAAGAGCTGT
GTTCGTTGATCTCGAACCAACTGTTGTTGATGAGGTAAGAACTGGAACAT
ACCGCCAGTTGTTCCACCCTGAACAATTGATCACTGGTAAGGAAGATGCC
GCCAACAACTACGCACGTGGACACTACACTATCGGAAAAGAGATTGTTG
ATGTTGTTTTGGACCGAATCAGGAAATTGGCTGATCAGTGCACTGGTCTT
CAAGGTTTCCTGATCTTCCACTCTTTCGGAGGTGGTACTGGATCTGGTTTC
ACATCTTTGTTGATGGAAAGACTCAGCGTTGACTACGGAAAGAAGAGTAA
ATTAGAATTCGCCATCTACCCAGCCCCTCAAGTATCCACAGCTGTAGTTG
AGCCATACAACTCCATCTTGACCACACATACAACTCTTGAACACAGTGAC
TGTGCATTCATGGTCGATAATGAAGCCATCTATGACATCTGCCGTCGTAA
TCTCGATATTGAACGTCCAACTTACACTAACTTGAATCGTCTTATTGGCCA
GATTGTTTCTTCAATCACAGCTTCTCTCCGTTTCGATGGTGCCCTCAATGT
TGACTTGACTGAATTCCAGACCAATTTGGTCCCATACCCCCGTATTCATTT
CCCATTGGTCACCTATGCACCAGTCATCTCCGCTGAAAAGGCTTACCATG
AACAATTGTCCGTATCAGAAATCACTAACGCTTGTTTTGAACCAGCCAAC
CAAATGGTGAAATGTGATCCACGTCATGGCAAATACATGGCTTGTTGCAT
GTTGTACCGTGGTGATGTTGTACCCAAAGACGTCAACGCTGCCATTGCTT
CCATCAAGACCAAGAGAACAATTCAGTTTGTTGACTGGTGTCCAACTGGT
TTCAAAGTTGGTATCAACTACCAACCCCCAACCGTGGTACCCGGTGGTGA
CTTGGCTAAGGTACAACGTGCCGTCTGCATGTTGTCCAACACTACAGCTA
TTGCTGAAGCTTGGGCTAGGTTGGACCACAAGTTCGACTTGATGTACGCC
AAACGTGCTTTCGTCCATTGGTATGTTGGAGAAGGTATGGAAGAAGGAGA
ATTCTCTGAAGCTCGTGAGGATTTGGCTGCTCTAGAGAAAGATTACGAAG
AGGTTGGCATGGACTCCGTCGAAGGCGAAGGCGAAGGTGGTGAAGAATA
C
(SEQ ID NO.: 24)
TAATACGACTCACTATAGGGAGATCGCCATCTACCCAGCCCCTCAAG
TATCCACAGCTGTAGTTGAGCCATACAACTCCATCTTGACCACACATACA
ACTCTTGAACACAGTGACTGTGCATTCATGGTCGATAATGAAGCCATCTA
TGACATCTGCCGTCGTAATCTCGATATTGAACGTCCAACTTACACTAACTT
GAATCGTCTTATTGGCCAGATTGTTTCTTCAATCACAGCTTCTCTCCGTTT
CGATGGTGCCCTCAATGTTGACTTGACTGAATTCCAGACCAATTTGGTCC
CATACCCCCGTATTCATTTCCCATTGGTCACCTATGCACCAGTCATCTCCG
CTGAAAAGGCTTACCATGAACAATTGTCCGTATCAGAAATCACTAACGCT
TGTTTTGAACCAGCCAACCAAATGGTGAAATGTGATCCACGTCATGGCAA
ATACATGGCTTGTTGCATGTTGTACCGTGGTGATGTTGTACCCAAAGACG
TCAACGCTGCCATTGCTTCCATCAAGACCAAGAGAACAATTCAGTTTGTT
GACTGGTGTCCAACTGGTTTCAAAGTTGGTATCAACTACCAACCCCCAAC
CGTGGTACCCGAGAGGGATATCACTCAGCATAAT

DS9 gene

As used herein, the terms “DS9 gene”, “ADP/ATP translocase 3-like”, and “ADP/ATP carrier protein (AAC)” can be used interchangeably, and are responsible for transporting phosphorylated synthesis of ATP to the cytoplasm as the main ability supply of cells, providing power for thermodynamic reaction, which is expressed in the nymph stage.

In the present invention, this protein is a transport protein that allows the intracellular exchange of adenosine diphosphate (ADP) and mitochondrial adenosine triphosphate (ATP) to cross the mitochondrial inner membrane. Free ADP is transported from the cytoplasm to the mitochondrial matrix, while ATP produced by oxidative phosphorylation is transported from the mitochondrial matrix to the cytoplasm, thereby providing the cell with the main energy.

In one embodiment of the present invention, based on the RNAi technology, the DS9 gene is used as a target to screen the RNA fragments against the DS9 gene. Preferably, the sequence of the DS9 gene fragment is shown in SEQ ID NO: 2 or 25:

(SEQ ID NO.: 2)
ATGGCCGAAACCAAAGCGCCGAAGGACCCGTATGGTTTCTTGAAGG
ACTTCATGGCCGGTGGTATCTCCGCTGCCGTGTCGAAGACCGCCGTGGCT
CCGATCGAGCGCGTCAAGCTTATCCTGCAAGTGCAGGCCGCTTCCACGCA
GATCGCCGCCGACCAACAGTACAAAGGAATTATGGACTGTTTGGTGAGA
ATCCCAAAAGAACAAGGATTTGCCAGTTTCTGGAGAGGTAACTTTGCCAA
TGTCATCAGGTACTTCCCAACACAAGCATTGAACTTTGCTTTCAAGGATG
TCTACAAACAGGTGTTTATGGACGGTGTGGATAAAAAGACTCAATTCTGG
CGGTATTTTGCTGGTAACTTGGCATCTGGTGGTGCTGCTGGAGCAACATC
TTTGTGCTTTGTATACCCCCTCGATTACGCACGTACACGATTAGGAGCTGA
TGTCGGTAAAGGACCAGCTGAAAGGCAGTTCAAAGGTCTTGGTGATTGTT
TAGCCAAAACCGTCAAGTCTGATGGTCCCATTGGTTTGTACCGTGGTTTC
ATTGTATCAGTACAGGGTATCATCATCTACCGTGCTGCATACTTTGGATTT
TTCGACACAGCTAAGGGAATGTTGCCAGACCCCAAGAATACTCCATTCTT
AGTTTCATGGGGTATCGCCCAATTTGTAACAACATTCGCTGGTATTATGTC
CTATCCATTTGACACAGTCAGACGTCGTATGATGATGCAATCTGGCCGTG
CTGCTGACCAACGCATGTACAAGAGCACATTGGACTGCTGGGGTAAACTT
TACAAGAATGAAGGTACATCTGCTTTCTTCAAGGGTGCATTCTCCAACGT
ACTCAGAGGTACTGGTGGTGCCTTGGTGTTGGTCTTCTACGACGAACTCA
AAAACCTCATG
(SEQ ID NO.: 25)
TAATACGACTCACTATAGGGAGAGCCGGTGGTATCTCCGCTGCCGTG
TCGAAGACCGCCGTGGCTCCGATCGAGCGCGTCAAGCTTATCCTGCAAGT
GCAGGCCGCTTCCACGCAGATCGCCGCCGACCAACAGTACAAAGGAATT
ATGGACTGTTTGGTGAGAATCCCAAAAGAACAAGGATTTGCCAGTTTCTG
GAGAGGTAACTTTGCCAATGTCATCAGGTACTTCCCAACACAAGCATTGA
ACTTTGCTTTCAAGGATGTCTACAAACAGGTGTTTATGGACGGTGTGGAT
AAAAAGACTCAATTCTGGCGGTATTTTGCTGGTAACTTGGCATCTGGTGG
TGCTGCTGGAGCAACATCTTTGTGCTTTGTATACCCCCTCGATTACGCACG
TACACGATTAGGAGCTGATGTCGGTAAAGGACCAGCTGAAAGGCAGTTC
AAAGGTCTTGGTGATTGTTTAGCCAAAACCGTCAAGTCTGATGGTCCCAT
TGGTTTGTACCGTGGTTTCATTGTATCAGTACAGGGTATCATCATCTACCG
TGCTGCATACTTTGGATTTTTCGACACAGCTAAGGGAATGTTGCCAGACC
CCA AGAGGGATATCACTCAGCATAAT

DS15 Gene

As used herein, the terms “DS15 gene”, “heat shock protein 83-like”, and “heat shock protein 83” can be used interchangeably. They are intracellular molecular chaperone proteins that play an important role in protein interactions, such as assisting in folding and assisting in the establishment of a suitable protein conformation, which is expressed in the nymph stage.

In the present invention, heat shock proteins (HSP) are a family of proteins produced by cells in response to exposure to stress conditions. They are first associated with heat shock, but are now known in other stresses, including exposure to cold, and in wound healing or tissue remodeling. Many members of this group perform chaperone molecular functions by stabilizing new proteins to ensure proper folding or by helping to fold proteins damaged by cellular stress. Increasement is the regulation of transcription. The significant up-regulation of heat shock proteins is a key part of the heat shock response, which is mainly induced by heat shock factor (HSF).

In one embodiment of the present invention, based on RNAi technology, the DS15 gene is used as a target to screen RNA fragments against the DS15 gene. Preferably, the sequence of the DS15 gene fragment is shown in SEQ ID NO: 3 or 26:

(SEQ ID NO.: 3)
ATGCCTGAAGACGTTACCATGACTGCATCTGATGATGTTGAGACCTT
CGCTTTCCAAGCTGAGATCGCTCAGCTTATGTCCCTCATCATCAACACCTT
CTACTCGAACAAAGAAATCTTTTTGCGAGAATTGGTATCCAATTCTTCTG
ATGCATTGGACAAAATTCGTTATGAGTCATTGACTGATCCATCCAAATTG
GAATCTGGCAAAGATTTACACATTAAAATCATCCCCAATGCGGAAGAAA
AAACTCTGACCATTATTGACACTGGTATCGGTATGACCAAAGCTGATCTA
GTCAACAACTTGGGAACCATTGCTAAATCTGGTACTAAGGCTTTCATGGA
AGCTTTACAAGCTGGAGCTGATATTTCCATGATTGGTCAATTTGGTGTGG
GTTTCTATTCCGCCTATCTGGTAGCTGACAAAGTCACTGTTGTTTCCAAAC
ACAACGACGATGAACAATATTTGTGGGAATCTGCTGCCGGAGGTTCATTC
ACCATCCGTACTGATCCTGGTGAACCATTGGGCCGTGGTACCAAAATTGT
CCTTCAAATCAAAGAAGATCAAGCTGAGTTCCTCCAACAAGAAAAAATTA
CCAGCATCATCAAGAAGCACTCTCAATTCATTGGCTACCCAATCAAATTA
ATCGTTGAGAATGAACGTACCAAAGAAGTCAGCGATGATGAAGCTGAAG
AAGAAAAGAAAGATGAAGTTGAAGGTGAAACTGAAGAAGACAAAAAAC
CCAAAATTGAGGATGTTGGTGAGGATGAAGACGAAGACAAAAAAGATGA
AGACAAAGACAAAAAGAAGAAGAAGACTATTAAAGAAAAGTACTTGGAT
GAAGAGGTCTTGAACAAGACAAAACCAATCTGGACACGCAACCCTGATG
ATATCAGCCAAGATGAATATGGTGAATTCTACAAATCCTTAACCAATGAC
TGGGAAGATCATTTAGCCGTCAAACATTTCTCTGTGGAAGGACAACTTGA
ATTCAGAGCATTGTTATTCATTCCCAAGCGTGCGCCTTATGACATGTTTGA
GAACAAGAAGAAGAAGAACAACATTAAATTATATGTCCGTCGTGTCTTCA
TCATGGACAACTGCGAAGACCTCATGCCAGAATACTTGAACTTCATCAAG
GGTGTTGTTGACAGTGAGGATTTGCCGTTGAACATCTCCCGTGAAATGCT
CCAACAAAACAAGATCTTGAAAGTTATCAGGAAGAATTTGGTTAAGAAA
TGTTTGGAATTGTTCGAGGAATTGGCTGAAGACAAGGACAACTACAAGA
AATTGTACGAACAGTTCAGCAAGAACTTGAAACTTGGAATCCACGAAGAT
AGCCAAAACAGAAAGAAACTCTCAGACTTGTTGAGATTCCACTCCTCAGC
CAGTGGTGACGAATCATGCTCCCTTAAGGAGTATGTTGCACGTATGAAGC
CAAATCAAACCCACATTTACTACATCACAGGTGAAAGCCGTGAACAAGTA
TCCAACTCTTCATTCGTTGAACGTGTCAAGAAACGTGGTTTTGAAGTTATT
TACATGACTGAACCCATTGATGAATACGTTGTCCAACAAATGAAAGAATA
TGACGGCAAGAACTTGGTATCTGTCACTAAAGAAGGTTTGGACTTGCCTG
AAACCGATGAAGAAAAGAAGAAGCGCGAGGATGATCAATCCAGATTTGA
AAAATTGTGCAAAGTTGTTAAGGACATTTTGGACAAGAAAGTTGAGAAG
GTTGTCATCAGTAACAGACTTGTTGAGTCTCCCTGTTGCATTGTCACATCT
CAGTATGGTTGGACTGCCAACATGGAACGTATCATGAAGGCACAAGCACT
CAGAGATTCATCTACCATGGGTTATATGTCTGCCAAAAAACACTTGGAAA
TCAACCCTGACCACCCGATCATTGAAACACTCAGACAAAAGGCTGAAGCT
GATTGCAACGACAAGGCTGTCAGAGACTTGGTCATGCTTTTGTTCGAGAC
AAGTTTGTTGTCATCTGGTTTTGGACTTGAAGACCCACAAGTTCACGCTTC
TAGAATCCACAGAATGATCAAATTGGGTTTGGGCATTGATGAAGATTTGC
CAGTAGTTGAAGAAAAATCTGCTGAAGTTGAAGCCTCCGAGCCTGTTGTT
GAAGCTGATGCTGAAGATTCTTCTCGCATGGAAGAAGTTGAT
(SEQ ID NO.: 26)
TAATACGACTCACTATAGGGAGATGGTGAACCATTGGGCCGTGGTAC
CAAAATTGTCCTTCAAATCAAAGAAGATCAAGCTGAGTTCCTCCAACAAG
AAAAAATTACCAGCATCATCAAGAAGCACTCTCAATTCATTGGCTACCCA
ATCAAATTAATCGTTGAGAATGAACGTACCAAAGAAGTCAGCGATGATG
AAGCTGAAGAAGAAAAGAAAGATGAAGTTGAAGGTGAAACTGAAGAAG
ACAAAAAACCCAAAATTGAGGATGTTGGTGAGGATGAAGACGAAGACAA
AAAAGATGAAGACAAAGACAAAAAGAAGAAGAAGACTATTAAAGAAAA
GTACTTGGATGAAGAGGTCTTGAACAAGACAAAACCAATCTGGACACGC
AACCCTGATGATATCAGCCAAGATGAATATGGTGAATTCTACAAATCCTT
AACCAATGACTGGGAAGATCATTTAGCCGTCAAACATTTCTCTGTGGAAG
GACAACTTGAATTCAGAGCATTGTTATTCATTCCCAAGCGTGCGCCT
AGAGGGATATCACTCAGCATAAT

DS25 Gene

As used herein, the terms “DS25 gene”, “eukaryotic initiation factor 4A-like”, and “eukaryotic initiation factor complex type of 4A” can be used interchangeably, it is a helicase that unwinds double-stranded RNA and is also a functional protein necessary for ribosomal subunit binding, which is expressed in the nymph stage.

In the present invention, the eukaryotic initiation factor complex forms a ternary complex with GTP and the initiator Met-tRNA. This process is regulated by guanine nucleotide exchange and phosphorylation, and is the main regulatory element of the bottleneck of gene expression. Before the translation progresses to the extension stage, many initiation factors must promote the synergy of ribosomes and mRNA, and ensure that the 5′UTR of the mRNA is sufficiently lacking in secondary structure. The fourth group of eukaryotic initiation factors promotes this combination; it is of significance in the normal regulation of translation and the transformation and progression of cancer cells.

In one embodiment of the present invention, based on RNAi technology, the DS25 gene is used as a target to screen RNA fragments against the DS25 gene. Preferably, the sequence of the DS25 gene fragment is shown in SEQ ID NO: 4 or 27:

(SEQ ID NO.: 4)
ATGAATGCTAATGAGACGAAAAATGGACCTCCTAGTGAAACCAATG
ACTACTCGGGACCACCTGGCATGGACGTCGGTGGAACTATTGAGTCTGAC
TGGAAAGAAGTGGTGGATAACTTTGATGAGATGAATTTAAAAGAAGAAT
TGTTGCGTGGTATTTATGGATATGGTTTTGAAAAGCCATCAGCTATTCAAC
AACGTGCTATTTTGCCGTGCATCAAGGGACATGATGTCATTGCTCAGGCC
CAATCTGGTACTGGCAAGACAGCTACTTTTTCCATTTCTATTCTCCAACAA
ATTGATACAAGTTTGAATGAGTGCCAAGCACTTATTTTGGCACCAACACG
TGAATTGGCTCAACAGATTCAAAAGGTGGTCATTGCTTTGGGTGATTTCA
TGAAAGCTGATTGTCATGCTTGCATTGGCGGTACAAACGTTCGTGATGAC
ATGCGTAAGCTGGATACTGGATCCCATGTAGTTGTTGGAACTCCTGGCCG
TGTTTATGACATGATTGCTAGAAAATCCCTAAGAACTCAATTTATCAAGA
TATTTGTGTTGGACGAAGCTGATGAAATGTTGTCTCGAGGTTTCAAAGAT
CAAATTAAAGAGGTGTTCAAGTTCCTCGAAGAAGATATTCAGGTCATTCT
GTTGTCTGCTACAATGCCCGAGGACGTTTTGGATGTGAGCACTCATTTCAT
GCGTAATCCAGTACGCATTCTTGTTCAAAAGGAAGAACTGACATTGGAAG
GTATCAAACAGTTTTACATCAATGTTACCAAAGAAGAATGGAAGTTTGAC
ACTCTATGTGATTTGTACGACACTCTTAGTATCACCCAGGCTGTGATCTTC
TGTAACACACGTCGTAAGGTAGAGTGGTTGACTGAAAATATGCGTTTGAA
AACATTTACTGTATCAGCTATGCATGGAGAAATGGACCAACGTCAACGTG
AGCTAATTATGCGTCAATTCCGTTCTGGCTCTAGTCGTGTTCTAATTACCA
CTGATTTGTTGGCTCGAGGCATTGATGTACAACAAGTTTCTCTGGTCATCA
ATTACGATTTGCCGTCCAATCGTGAAAACTATATTCACAGGATTGGACGT
TCTGGCCGTTTCGGTCGTAAAGGAGTCGCCATTAATTTTATCACCGAAGA
CGACAAAAGAGCTATGAAGGATATTGAATCATTTTACAACACTCACGTGC
TCGAGATGCCACAGAATGTGGCCGATTTGCTG
(SEQ ID NO.: 27)
TAATACGACTCACTATAGGGAGACCACCTGGCATGGACGTCGGTGG
AACTATTGAGTCTGACTGGAAAGAAGTGGTGGATAACTTTGATGAGATGA
ATTTAAAAGAAGAATTGTTGCGTGGTATTTATGGATATGGTTTTGAAAAG
CCATCAGCTATTCAACAACGTGCTATTTTGCCGTGCATCAAGGGACATGA
TGTCATTGCTCAGGCCCAATCTGGTACTGGCAAGACAGCTACTTTTTCCAT
TTCTATTCTCCAACAAATTGATACAAGTTTGAATGAGTGCCAAGCACTTA
TTTTGGCACCAACACGTGAATTGGCTCAACAGATTCAAAAGGTGGTCATT
GCTTTGGGTGATTTCATGAAAGCTGATTGTCATGCTTGCATTGGCGGTAC
AAACGTTCGTGATGACATGCGTAAGCTGGATACTGGATCCCATGTAGTTG
TTGGAACTCCTGGCCGTGTTTATGACATGATTGCTAGAAAATCCCTAAGA
ACTCAATTTATCAAGATATTTGTGTTGGACGAAGCTGATGAAATGTTGTC
TCGAGGTTTCAAAGATCAAATTAAAGAGGTGTTCAAGTTCCTCGAAGAAG
ATATTCAGGTCATTCTGTTGTCTGCTACAATGCCCGAGGACGT
AGAGGGATATCACTCAGCATAAT

DS27 Gene

As used herein, the terms “DS27”, “troponin T-like isoform 3”, and “troponin type 3” can be used interchangeably. It mediates Ca ion channels and regulates the contraction regulation function of insect striated muscle, which is expressed in the nymph and adult stages.

In the present invention, troponin is attached to the protein tropomyosin and is located in the grooves between actin filaments in muscle tissue. In a relaxed muscle, tropomyosin blocks the attachment site of the myosin cross bridge, thereby preventing contraction. When muscle cells are stimulated to contract by an action potential, calcium channels open in the sarcoplasm membrane and release calcium into the sarcoplasm. Some of this calcium attaches to troponin, causing it to change shape, exposing the binding site of myosin (active site) on actin filaments. The binding of myosin to actin causes cross bridges to form and start to contract muscles.

Troponin activation. Troponin C (red) binds to Ca2⁺ and stabilizes the activated state, wherein troponin I (yellow) no longer binds to actin. Troponin T (blue) fixes the complex to tropomyosin.

Troponin is found in skeletal muscle and heart muscle, but the specific version of troponin differs in different types of muscles. The main difference is that the TnC subunit of troponin has four calcium binding sites in skeletal muscle, but only three in cardiac muscle. Opinions on the actual content of calcium bound to troponin vary from expert to source.

In one embodiment of the present invention, based on the RNAi technology, the DS27 gene is used as a target to screen the RNA fragments against the DS27 gene. Preferably, the sequence of the DS27 gene fragment is shown in SEQ ID NO: 5 or 28:

(SEQ ID NO.: 5)
ATGTCCGACGAAGAAGAAGTGTACACTGATTCCGAAGAAGAAACGC
AACCGGAGCCTGAAAAAAGCAAAGATGGAGATGGAGATCCCGAATTCGT
TAAGAGGCAAGAATTAAAATCTTCAGCCTTAGACGAACAGCTTAAAGAG
TACATCCAAGAATGGCGCAAACAGCGGTCAAAGGAAGAAGACGACTTAA
AGAAGTTGAAGGAAAAACAGGCCAAGCGCAAGGTTATGCGAGCGGAAG
AAGAGAAGAGAATGGCCGAGAGAAAGAAGCAAGAAGAAGAACGCAGAC
AGAGAGAAGTCGAGGAAAAGAAACAAAAGGACATCGAAGAAAAACGTA
AACGTCTAGAAGAGGCCGAGAAAAAACGGCAAGCTATGATGGCTGCTCT
TAAGGAACAAACCAATAAATCTAAAGGACCAAATTTCACCATCAGCAAA
AAAGAAGGTGCGTTGAGTATGACTTCTGCCCAACTTGAACGCAATAAAAC
CAGAGAACAGATCGAAGAAGAAAAGAAAATATCGTTGAGCTTCAGAATC
AAACCTTTGAATATTGAAGGATTCTCTGTGCAAAAACTCCAATTCAAAGC
TACCGAACTCTGGGACCAGATCATCAAGTTGGAAACAGAAAAATACGAT
TTGGAGGAAAGGCAAAAGAGACAAGATTACGACTTGAAAGAGTTGAAAG
AACGTCAGAAGCAACAACTCCGCCACAAGGCTCTGAAGAAAGGTCTCGA
CCCCGAAGCCCTAACCGGCAAATACCCACCCAAGATCCAAGTCGCTTCCA
AGTACGAGAGGCGAGTTGACACGAGGTCTTATGATGACAAAAAGAAGCT
GTTCGAAGGAGGTTATATGGAAACCACTAAAGAATCAATGGAAAAACAA
TGGACAGAAAAAAGTGACCAATTCGGTGGCCGCGCTAAAGGACGATTAC
CGAAATGGTTCGGCGAACGTCCGGGCAAGAAGAAGGATGACCCAGACAC
ACCCGAAGAGGAAGAGCTCAAGAAAAACGAGGAAGACGAAGAACCGTT
TGGCCTCGACGACGAAGAAGCTGAAGAAGAAGTTGAAGAGGAAGAAGA
GGAGGAAGAAGAAGAGGAAGAGGAGGAGGAAGAGGAAGAAGAGGAAG
AAGAAGAAGAGGAAGAGGAAGAAGAAGAAGAA
(SEQ ID NO.: 28)
TAATACGACTCACTATAGGGAGAGCGCAAGGTTATGCGAGCGGAAG
AAGAGAAGAGAATGGCCGAGAGAAAGAAGCAAGAAGAAGAACGCAGAC
AGAGAGAAGTCGAGGAAAAGAAACAAAAGGACATCGAAGAAAAACGTA
AACGTCTAGAAGAGGCCGAGAAAAAACGGCAAGCTATGATGGCTGCTCT
TAAGGAACAAACCAATAAATCTAAAGGACCAAATTTCACCATCAGCAAA
AAAGAAGGTGCGTTGAGTATGACTTCTGCCCAACTTGAACGCAATAAAAC
CAGAGAACAGATCGAAGAAGAAAAGAAAATATCGTTGAGCTTCAGAATC
AAACCTTTGAATATTGAAGGATTCTCTGTGCAAAAACTCCAATTCAAAGC
TACCGAACTCTGGGACCAGATCATCAAGTTGGAAACAGAAAAATACGAT
TTGGAGGAAAGGCAAAAGAGACAAGATTACGACTTGAAAGAGTTGAAAG
AACGTCAGAAGCAACAACTCCGCCACAAGGCTCTGAAGAAAGGTCTCGA
CCCCGAAGCCCTAACCGAGAGGGATATCACTCAGCATAAT

DS45 Gene

As used herein, the terms “DS45 gene”, “Y-box protein Ct-p40-like”, and “y box binding protein Ct-p40-like” can be used interchangeably, and affect cell differentiation and cytoskeleton formation. Deletion of it will inhibit signal transduction pathways inside and outside the cell, involved in DNA damage repair and transcription, and it is expressed in the nymph stage.

In one embodiment of the present invention, based on RNAi technology, the DS45 gene is used as a target to screen RNA fragments directed against the DS45 gene. Preferably, the sequence of the DS45 gene fragment is shown in SEQ ID NO: 6 or 29:

(SEQ ID NO.: 6)
ATGGCGGAACAAGTCGGCGAGAGGAGGACGGAACGGCCGCCGCAG
AAGCCCGTGGCCCAAAAGCCGGTCATATCTGTGAAAGTCACCGGCGTTGT
TAAATGGTTCAACGTCAAAAGCGGTTATGGTTTTATTAATCGTAATGATA
CAAAAGAAGATATATTTGTACATCAGTCTGCTATTATCAAGAACAACCCT
AAGAAAATTGTACGCAGTGTCGGTGATGGAGAAACTGTAGAATTTGACGT
TGTTGAGGGCGAAAAAGGTCACGAAGCAGCAAATGTTACTGGTCCAGAT
GGAGAAGCTGTTAAAGGATCACCTTATGCAGCTGAAAGAAGAAGAAATA
ACTATCGTCAGTGGTTTTATGGACGCCGTCCTAATACCCGTCCAAGAAAT
GGTGGTCAACCTCCAAGAGATGGTAGTCCAAGTGGTGACAAGGAAGAAA
CTGAAAATGAAGTAGGAGAACAACCAAGACGTTACCGCCAGCCACGTCA
ACAGAATTGGTATAATAGCTATCGTGGAAATCGAAGAGGTCCACCACCA
AATAGAGGAGAAGGTGGTGATTACAATGGTGGAGATAATTATGGATATG
ATAGTTCACCTCCTGGTAGAGGCAGAGGTCGTGGGATGGGTGCGCCTAGA
CGTTTCTTTAGACGTGGCAGTGGATTTAGAGGGAGCCGTGGAACAGGTGG
TCCACCCAGAAGACCATATCAAGATGAAAATCAGGACAATGAATATAAT
CAAAGTGATGAAAATGGAGCAAATAGACCTCGTCCTCGCTATCGCCGCCG
CAATAATCGTTCTAGAGCGAGAAGTGATGGTCCTCCAAGAGCCAATAGCC
AAAGTGACAATGAATCTAAACAAAAAAACTTTGGAGGAGAAGCATTGGA
ACTGGATGAAAGTAGTCATGCT
(SEQ ID NO.: 29)
TAATACGACTCACTATAGGGAGAGCAGAAGCCCGTGGCCCAAAAGC
CGGTCATATCTGTGAAAGTCACCGGCGTTGTTAAATGGTTCAACGTCAAA
AGCGGTTATGGTTTTATTAATCGTAATGATACAAAAGAAGATATATTTGT
ACATCAGTCTGCTATTATCAAGAACAACCCTAAGAAAATTGTACGCAGTG
TCGGTGATGGAGAAACTGTAGAATTTGACGTTGTTGAGGGCGAAAAAGG
TCACGAAGCAGCAAATGTTACTGGTCCAGATGGAGAAGCTGTTAAAGGA
TCACCTTATGCAGCTGAAAGAAGAAGAAATAACTATCGTCAGTGGTTTTA
TGGACGCCGTCCTAATACCCGTCCAAGAAATGGTGGTCAACCTCCAAGAG
ATGGTAGTCCAAGTGGTGACAAGGAAGAAACTGAAAATGAAGTAGGAGA
ACAACCAAGACGTTACCGCCAGCCACGTCAACAGAATTGGTATAATAGCT
ATCGTGGAAATCGAAGAGGTCCACCACCAAATAGAGGAGAAGGTGGTGA
TTACAATGGTGGAGATAATTATGGATATGATAGTTCACCTCCTGGTAGAG
GCAGAGGTCGTGGGATGGGTGCGCCTAAGAGGGATATCACTCAGCATAA
T

dsRNA Construct and its Application

The present invention provides a dsRNA construct. The dsRNA construct is double-stranded, and its positive or negative strand contains a structure as shown in Formula I:

Seq_{forward}-X-Seqᵣₑᵥₑᵣₛₑ Formula I

wherein

Seq_{forward} is a nucleotide sequence of insect nymph and/or adult stage regulation-related gene or fragment;

Seqᵣₑᵥₑᵣₛₑ is a nucleotide sequence that is basically complementary to Seq_{forward};

X is an intervening sequence located between the Seq_{forward} and the Seqᵣₑᵥₑᵣₛₑ, and the intervening sequence is not complementary to the Seq_{forward} and the Seqᵣₑᵥₑᵣₛₑ,

wherein the insect nymph and/or adult stage regulation-related gene is selected from the group consisting of DS7 gene, DS9 gene, DS15 gene, DS25 gene, DS27 gene, DS45 gene and a combination thereof.

In a preferred embodiment of the present invention, the length of the Seq_{forward} and Seqᵣₑᵥₑᵣₛₑ is at least 50 bp.

In a preferred embodiment of the present invention, the dsRNA construct is ingested by insects (such as aphids) to form a dsRNA of Formula II,

[chemical structure]

wherein

Seq′_{forward} is a RNA sequence or sequence fragment corresponding to the Seq_{forward} sequence;

Seq′ᵣₑᵥₑᵣₛₑ is a sequence that is basically complementary to the Seq′_{forward};

X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ,

∥ represents the hydrogen bond formed between Seq_{forward} and Seqᵣₑᵥₑᵣₛₑ.

The present invention also provides the use of the dsRNA construct, which is used to: (1) improve the control effect of aphids; and/or (2) increase the dropping rate of insect population; and/or (3) reduce the expression level of nymph and/or adult stage regulation-related gene; (4) reduce the initial number of insect population; and/or (5) reduce the damage rate of plants; and/or (6) reduce the damage degree of crops and improve the quality of crop products.

dsRNA and its Applications

The present invention also provides a dsRNA as shown in Formula II,

[chemical structure]

wherein

Seq′_{forward} is a RNA sequence or sequence fragment corresponding to Seq′_{forward} sequence;

Seq′ᵣₑᵥₑᵣₛₑ is a sequence that is basically complementary to the Seq′_{forward};

X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ;

∥ represents the hydrogen bond formed between Seq_{forward} and Seqᵣₑᵥₑᵣₛₑ.

In another preferred embodiment, the length of the intervening sequence X is 0-300 bp, preferably 100 bp.

The insect nymph and/or adult stage regulation-related gene is derived from aphids; the sequence of the DS7 gene is shown in SEQ ID NO. 1; the sequence of the DS9 gene is shown in SEQ ID NO. 2; The sequence of the DS15 gene is shown in SEQ ID NO. 3; the sequence of the DS25 gene is shown in SEQ ID NO. 4; the sequence of the DS27 gene is shown in SEQ ID NO. 5; the sequence of the DS45 gene is shown in SEQ ID NO. 6.

In another preferred embodiment, the insects are phytophagous insects, preferably from Hemiptera insects, and most preferably from Aphis.

The present invention also provides the use of the dsRNA, which is used to: (1) improve the control effect of aphids; and/or (2) increase the dropping rate of insect population; and/or (3) reduce the expression level of nymph and/or adult stage regulation-related gene; and/or (4) reduce the initial number of insect population; and/or (5) reduce the damage rate of plants.

Composition and its Application

The present invention also provides a composition. In response to the problem of efficiently killing aphids, the inventor has developed RNAi fragments for target genes based on RNAi technology, and improved the control effect of aphids and the dropping rate of insect population by feeding insects or spraying insects directly, making RNAi have the effect of inhibiting gene expression, and finally achieving the purpose of efficiently killing aphids. The method of the present invention is efficient, convenient, fast, accurate and pollution-free.

The composition includes a dsRNA construct and/or dsRNA, and an effective amount of a carrier acceptable for insect feeding. In another preferred embodiment, the composition is a composition used to induce or cause the death of aphid nymphs and/or adult stage.

In another preferred embodiment, the dsRNA has the following sequence:

dsRNA1: having a sequence corresponding to SEQ ID NO. 1 or 24;

dsRNA2: having a sequence corresponding to SEQ ID NO. 2 or 25;

dsRNA3: having a sequence corresponding to SEQ ID NO. 3 or 26;

dsRNA4: having a sequence corresponding to SEQ ID NO. 4 or 27;

dsRNA5: having a sequence corresponding to SEQ ID NO. 5 or 28;

dsRNA6: having a sequence corresponding to SEQ ID NO. 6 or 29.

The present invention also provides a use of the composition, which is selected from the following group:

(1) improving the control effect of aphids; and/or

(2) increasing the dropping rate of insect population; and/or

(3) decreasing the expression level of nymph and/or adult stage regulation-related gene; and/or

(4) reducing the initial number of insect population; and/or

(5) reducing plant damage rate; and/or

(6) reducing crop damage degree and improving the quality of crop products.

In a preferred embodiment of the present invention, the composition is an aqueous solution, and the pH is usually about 5-8, preferably, the pH is about 6-8.

As used herein, the term “effective amount” or “effective dose” refers to an amount that can produce function or activity for feeding the insect and can be accepted by the insect. Preferably, the content of dsRNA1 is about 1-500 ng/μl, preferably, 5-300 ng/μl, more preferably, 50-150 ng/μl; the content of dsRNA2 is about 1-500 ng/μl, preferably, 5-300 ng/μl, more preferably, 50-150 ng/μl; the content of dsRNA3 is about 1-500 ng/μl, preferably, 5-300 ng/μl, more preferably, 50-150 ng/μl; the content of dsRNA4 is about 1-500 ng/μl, preferably, 5-300 ng/μl, more preferably, 50-150 ng/μl; the content of dsRNA5 is about 1-500 ng/μl, preferably, 5-300 ng/μl, more preferably, 50-150 ng/μl; the content of dsRNA6 is about 1-500 ng/μl, preferably, 5-300 ng/μl, more preferably, 50-150 ng/μl. The selection of the preferred effective amount can be determined by a person of ordinary skill in the art according to various factors (for example, through a feeding experiment or a spray experiment).

As used herein, “insect feeding acceptable” ingredients are suitable for the insects without excessive adverse side effects (such as toxicity, irritation, and allergic reactions), that is, substances with a reasonable benefit/risk ratio.

As used herein, the term “carrier” includes various excipients and diluents. Such carriers include (but are not limited to): water, saline, buffer, glucose, glycerol, ethanol, and combinations thereof.

The composition of the present invention can be directly sprayed, fed, or made into an injection form, for example, prepared by conventional methods with water, physiological saline or an aqueous solution containing glucose and other adjuvants. The composition is preferably manufactured under sterile or RNase-free conditions.

The main advantages of the present invention include:

1) The dsRNA designed for specific target genes of the present invention can effectively kill aphids, improve the control effect of aphids (≥80%) and the dropping rate of insect population (≥70%);

2) The obtained dsRNA can be directly used to kill aphids and is convenient to use;

3) Low production cost, good stability, suitable for mass production;

4) Good environmental compatibility, green and pollution-free, and safe for humans and animals.

The invention will be further illustrated with reference to the following specific examples. It is to be understood that these examples are only intended to illustrate the invention, but not to limit the scope of the invention. For the experimental methods in the following examples without particular conditions, they are performed under routine conditions (e.g. Sambrook et al., Molecular Cloning: A Laboratory Manual (New York: Cold Spring Harbor Laboratory Press, 1989) or as instructed by the manufacturer. Unless otherwise specified, the materials and reagents used in the examples are all commercially available products.

General Methods and Materials

1. Aphid Breeding and Biological Testing

The green peach aphid (Myzus persicae) was cultivated and tested on radish seedlings cultivated in indoor greenhouses or plastic greenhouses, and the soybean aphid (Aphid glycine) was cultivated and tested on soybean seedlings cultivated in indoor greenhouses or plastic greenhouses. The temperature of the incubation room was 25±1° C., the relative humidity was 40-60%, and the photoperiod was 12 h: 12 h.

Before the test, a certain number of aphids were inoculated on the target plant and counted. After spraying a certain concentration of dsRNA, the counting was performed on day 1, 3, and 5 respectively. The test for each gene was repeated 10 times. According to the counting results, the control effect of the target gene was determined.

2. Statistical Methods of Control Effect

In this study, two statistical methods were used to evaluate the effect of target dsRNA on aphids.

The first method, the control effect, is calculated as follows:

Control effect (%)=(1−CK0×PT1/CK1×PT0)×100

wherein PT0: the number of insects before drug administration in the treatment area; PT1: the number of insects after drug administration in the treatment area;
CK0: the number of insects before drug administration in the control area; CK1: the number of insects after drug administration in the control area.

The second method, the dropping rate of insect population, is calculated as follows:

the dropping rate of insect population (%)=[(number of insects before drug administration−number of insects after drug administration)/number of insects before drug administration]×100

3. RNA Extraction and Quality Test

The total RNA was extracted using TRIzol® Reagent (Invitrogen), and the operation was performed according to the instructions: 1) adding 50-100 mg of Ostrinia nubilalis sample that was well ground into 1 mL of TRIzol, mixed well, and placed at room temperature for 5 minutes. 2) adding 200 μL of chloroform, shaked and mixed, and placed at room temperature for 3 minutes. 3) centrifuged at 12,000 rpm (4° C.) for 15 minutes, transferring the upper aqueous phase to another new centrifuge tube, adding 500 μL of pre-cooled isopropanol, shaked and mixed, and placed at room temperature for 10 minutes. 4) centrifuged at 12 000 rpm (4° C.) for 15 minutes, and carefully aspirating the supernatant. 5) washed with 500 μL of pre-cooled 75% ethanol and mixed gently with a vortex for 10 sec. 6) centrifuged at 12 000 rpm (4° C.) for 2 minutes, carefully aspirating the supernatant and drying it at room temperature for 5 minutes, adding an appropriate amount of DEPC sterilized water to dissolve it, and obtaining a total RNA sample. Detecting the absorbance under a spectrophotometer, detecting the total RNA quality by 1% agar gel electrophoresis, and storing it at −80° C., ready for use.

4. dsRNA Synthesis

Using the kit MEGAscript® RNAi Kit (Ambion) to synthesize dsRNA, and performing experimental operations according to the instructions. A T7 promoter sequence was added to the 5′end of the primer of the amplification template to facilitate subsequent dsRNA synthesis. Using pPigbac A3 EGFP as a template for the synthesis of control group dsEGFP, dsEGFP was used as a negative control to participate in the treatment of the experimental group in subsequent experiments. See Appendix S3 for the primers used to synthesize dsRNAs. During the synthesis process, template DNA and single-stranded RNA were removed with DNase and RNase, respectively.

5. Detection of Gene Expression (q-RT-PCR)

Using TRIzol® reagent (Invitrogen) for total RNA extraction, and the steps were strictly in accordance with the operation manual. Taking 1 μg of total RNA and using the kit ReverTra Ace® qPCR RT Master Mix with gDNA Remover (TOYOBO) to synthesize the first strand of cDNA. The kit used for the RT-qPCR reaction was SYBR® Premix Ex Taq™ II (Takara), and the primers were detailed in Appendix S3. For each gene sample, the detection was repeated 3 times, the expression level analysis selected the expression level of 18S rRNA for normalization. Data analysis was referred to 2^{-ΔΔCT} Method (Livak & Schmittgen, 2001). The corresponding value was obtained by calculating the mean value and standard error. In order to eliminate individual differences, the samples of each experimental group were a sample pool formed by 2 surviving larvae after treatment, and each experimental group was subjected to three biological replicates.

### Example 1 Target Gene Sequence and dsRNA Synthesis

In order to screen effective target genes of aphids based on RNA interference technology, transcriptome sequencing was performed on the green peach aphid (Myzus persicae) and the soybean aphid (Aphid glycine) (the sampling and sequencing analysis methods of the two aphids were same). After extracting total RNA from aphids at different developmental stages, the same amount of RNA was taken and mixed to form the total RNA for the entire developmental stage of aphids and sent to Shenzhen BGI Technology Services Co. LTD for transcriptome sequencing using the Illumina Hiseq2000 platform. After removing the adapters from the sequencing results, using the denove program to assemble, and then performing functional annotations on Unigene. In this study, target gene fragments were selected from these functionally annotated Unigenes for amplification and dsRNA was synthesized. Through a large number of screenings, the primers of the present invention for the amplification and synthesis of 6 target genes, the exogenous control gene GFP and the endogenous control gene DS50 from soybean aphid were shown in Table 1. The DNA sequences of the 6 gene fragments were shown in Table 2. Wherein ds7, ds9, and ds15 were against aphids, especially the green peach aphid, and ds25, ds27, and ds45 were against aphids, especially the soybean aphid.

TABLE 1
Amplification and synthesis of the primer
sequence of the target gene dsRNA.

| | | SEQ | | SEQ |
|---|---|---|---|---|
| | | ID | | ID |
| Name | Primer F | NO.: | Primer R | NO.: |
| dsGFP | TAATAC GACTCA CTATAG GGAGA | 7 | TAATAC GACTCA CTATAG GGAGA | 8 |
| | GACGAC GGCAAC TACA | | ACTCCA GCAGGA CCAT | |
| ds7 | TAATAC GACTCA CTATAG GGAGA | 9 | TAATAC GACTCA CTATAG GGAGA | 10 |
| | TCGCCA TCTACC CAGCCC CT | | CGGGTA CCACGG TTGGGG GT | |
| ds9 | TAATAC GACTCA CTATAG GGAGA | 11 | TAATAC GACTCA CTATAG GGAGA | 12 |
| | GCCGGT GGTATC TCCGCT GC | | TGGGGT CTGGCA ACATTC CCT | |
| ds15 | TAATAC GACTCA CTATAG GGAGA | 13 | TAATAC GACTCA CTATAG GGAGA | 14 |
| | TGGTGA ACCATT GGGCCG TGG | | AGGCGC ACGCTT GGGAAT GA | |
| ds25 | TAATAC GACTCA CTATAG GGAGA | 15 | TAATAC GACTCA CTATAG GGAGA | 16 |
| | CCACCT GGCATG GACGTC GG | | ACGTCC TCGGGC ATTGTA GCA | |
| ds27 | TAATAC GACTCA CTATAG GGAGA | 17 | TAATAC GACTCA CTATAG GGAGA | 18 |
| | GCGCAA GGTTAT GCGAGC GG | | CGGTTA GGGCTT CGGGGT CG | |
| ds45 | TAATAC GACTCA CTATAG GGAGA | 19 | TAATAC GACTCA CTATAG GGAGA | 20 |
| | GCAGAA GCCCGT GGCCCA AA | | TAGGCG CACCCA TCCCAC GA | |
| ds50 | TAATAC GACTCA CTATAG GGAGA | 21 | TAATAC GACTCA CTATAG GGAGA | 22 |
| | CGTGTC TGAGGC GGTTGC CA | | TGATCT TGGCCC GGAGAG CCGG | |

TABLE 2
Sequence fragments of 6 target genes.

| | | SEQ ID |
|---|---|---|
| Name | Sequence | NO.: |
| ds7 | >CL1054.Contig1_TY tubulin alpha | 1 |
| | chain-like | |
| | ATGCGTGAATGTATCTCTGTACACGTTGGCCAA | |
| | GCTGGTGTTCAAATCGGTAATGCCTGCTGGGAA | |
| | TTGTACTGTTTGGAACATGGAATTGCTCCAGAT | |
| | GGTCAAATGCCATCTGACAAGACCATTGGAGG | |
| | TGGAGACGACAGCTTCAACACCTTCTTCAGCGA | |
| | AACTGGCTCAGGCAAACATGTGCCAAGAGCTG | |
| | TGTTCGTTGATCTCGAACCAACTGTTGTTGATG | |
| | AGGTAAGAACTGGAACATACCGCCAGTTGTTCC | |
| | ACCCTGAACAATTGATCACTGGTAAGGAAGAT | |
| | GCCGCCAACAACTACGCACGTGGACACTACAC | |
| | TATCGGAAAAGAGATTGTTGATGTTGTTTTGGA | |
| | CCGAATCAGGAAATTGGCTGATCAGTGCACTG | |
| | GTCTTCAAGGTTTCCTGATCTTCCACTCTTTCGG | |
| | AGGTGGTACTGGATCTGGTTTCACATCTTTGTT | |
| | GATGGAAAGACTCAGCGTTGACTACGGAAAGA | |
| | AGAGTAAATTAGAATTCGCCATCTACCCAGCCC | |
| | CTCAAGTATCCACAGCTGTAGTTGAGCCATACA | |
| | ACTCCATCTTGACCACACATACAACTCTTGAAC | |
| | ACAGTGACTGTGCATTCATGGTCGATAATGAAG | |
| | CCATCTATGACATCTGCCGTCGTAATCTCGATA | |
| | TTGAACGTCCAACTTACACTAACTTGAATCGTC | |
| | TTATTGGCCAGATTGTTTCTTCAATCACAGCTTC | |
| | TCTCCGTTTCGATGGTGCCCTCAATGTTGACTTG | |
| | ACTGAATTCCAGACCAATTTGGTCCCATACCCC | |
| | CGTATTCATTTCCCATTGGTCACCTATGCACCA | |
| | GTCATCTCCGCTGAAAAGGCTTACCATGAACAA | |
| | TTGTCCGTATCAGAAATCACTAACGCTTGTTTT | |
| | GAACCAGCCAACCAAATGGTGAAATGTGATCC | |
| | ACGTCATGGCAAATACATGGCTTGTTGCATGTT | |
| | GTACCGTGGTGATGTTGTACCCAAAGACGTCAA | |
| | CGCTGCCATTGCTTCCATCAAGACCAAGAGAAC | |
| | AATTCAGTTTGTTGACTGGTGTCCAACTGGTTT | |
| | CAAAGTTGGTATCAACTACCAACCCCCAACCGT | |
| | GGTACCCGGTGGTGACTTGGCTAAGGTACAAC | |
| | GTGCCGTCTGCATGTTGTCCAACACTACAGCTA | |
| | TTGCTGAAGCTTGGGCTAGGTTGGACCACAAGT | |
| | TCGACTTGATGTACGCCAAACGTGCTTTCGTCC | |
| | ATTGGTATGTTGGAGAAGGTATGGAAGAAGGA | |
| | GAATTCTCTGAAGCTCGTGAGGATTTGGCTGCT | |
| | CTAGAGAAAGATTACGAAGAGGTTGGCATGGA | |
| | CTCCGTCGAAGGCGAAGGCGAAGGTGGTGAAG | |
| | AATAC | |
| ds9 | >CL3025.Contig1_TY ADP/ATP | 2 |
| | translocase 3-like | |
| | ATGGCCGAAACCAAAGCGCCGAAGGACCCGTA | |
| | TGGTTTCTTGAAGGACTTCATGGCCGGTGGTAT | |
| | CTCCGCTGCCGTGTCGAAGACCGCCGTGGCTCC | |
| | GATCGAGCGCGTCAAGCTTATCCTGCAAGTGCA | |
| | GGCCGCTTCCACGCAGATCGCCGCCGACCAAC | |
| | AGTACAAAGGAATTATGGACTGTTTGGTGAGA | |
| | ATCCCAAAAGAACAAGGATTTGCCAGTTTCTGG | |
| | AGAGGTAACTTTGCCAATGTCATCAGGTACTTC | |
| | CCAACACAAGCATTGAACTTTGCTTTCAAGGAT | |
| | GTCTACAAACAGGTGTTTATGGACGGTGTGGAT | |
| | AAAAAGACTCAATTCTGGCGGTATTTTGCTGGT | |
| | AACTTGGCATCTGGTGGTGCTGCTGGAGCAACA | |
| | TCTTTGTGCTTTGTATACCCCCTCGATTACGCAC | |
| | GTACACGATTAGGAGCTGATGTCGGTAAAGGA | |
| | CCAGCTGAAAGGCAGTTCAAAGGTCTTGGTGAT | |
| | TGTTTAGCCAAAACCGTCAAGTCTGATGGTCCC | |
| | ATTGGTTTGTACCGTGGTTTCATTGTATCAGTAC | |
| | AGGGTATCATCATCTACCGTGCTGCATACTTTG | |
| | GATTTTTCGACACAGCTAAGGGAATGTTGCCAG | |
| | ACCCCAAGAATACTCCATTCTTAGTTTCATGGG | |
| | GTATCGCCCAATTTGTAACAACATTCGCTGGTA | |
| | TTATGTCCTATCCATTTGACACAGTCAGACGTC | |
| | GTATGATGATGCAATCTGGCCGTGCTGCTGACC | |
| | AACGCATGTACAAGAGCACATTGGACTGCTGG | |
| | GGTAAACTTTACAAGAATGAAGGTACATCTGCT | |
| | TTCTTCAAGGGTGCATTCTCCAACGTACTCAGA | |
| | GGTACTGGTGGTGCCTTGGTGTTGGTCTTCTAC | |
| | GACGAACTCAAAAACCTCATG | |
| ds15 | >CL597.Contig1_TY heat shock | 3 |
| | protein 83-like | |
| | ATGCCTGAAGACGTTACCATGACTGCATCTGAT | |
| | GATGTTGAGACCTTCGCTTTCCAAGCTGAGATC | |
| | GCTCAGCTTATGTCCCTCATCATCAACACCTTCT | |
| | ACTCGAACAAAGAAATCTTTTTGCGAGAATTGG | |
| | TATCCAATTCTTCTGATGCATTGGACAAAATTC | |
| | GTTATGAGTCATTGACTGATCCATCCAAATTGG | |
| | AATCTGGCAAAGATTTACACATTAAAATCATCC | |
| | CCAATGCGGAAGAAAAAACTCTGACCATTATT | |
| | GACACTGGTATCGGTATGACCAAAGCTGATCTA | |
| | GTCAACAACTTGGGAACCATTGCTAAATCTGGT | |
| | ACTAAGGCTTTCATGGAAGCTTTACAAGCTGGA | |
| | GCTGATATTTCCATGATTGGTCAATTTGGTGTG | |
| | GGTTTCTATTCCGCCTATCTGGTAGCTGACAAA | |
| | GTCACTGTTGTTTCCAAACACAACGACGATGAA | |
| | CAATATTTGTGGGAATCTGCTGCCGGAGGTTCA | |
| | TTCACCATCCGTACTGATCCTGGTGAACCATTG | |
| | GGCCGTGGTACCAAAATTGTCCTTCAAATCAAA | |
| | GAAGATCAAGCTGAGTTCCTCCAACAAGAAAA | |
| | AATTACCAGCATCATCAAGAAGCACTCTCAATT | |
| | CATTGGCTACCCAATCAAATTAATCGTTGAGAA | |
| | TGAACGTACCAAAGAAGTCAGCGATGATGAAG | |
| | CTGAAGAAGAAAAGAAAGATGAAGTTGAAGGT | |
| | GAAACTGAAGAAGACAAAAAACCCAAAATTGA | |
| | GGATGTTGGTGAGGATGAAGACGAAGACAAAA | |
| | AAGATGAAGACAAAGACAAAAAGAAGAAGAA | |
| | GACTATTAAAGAAAAGTACTTGGATGAAGAGG | |
| | TCTTGAACAAGACAAAACCAATCTGGACACGC | |
| | AACCCTGATGATATCAGCCAAGATGAATATGGT | |
| | GAATTCTACAAATCCTTAACCAATGACTGGGAA | |
| | GATCATTTAGCCGTCAAACATTTCTCTGTGGAA | |
| | GGACAACTTGAATTCAGAGCATTGTTATTCATT | |
| | CCCAAGCGTGCGCCTTATGACATGTTTGAGAAC | |
| | AAGAAGAAGAAGAACAACATTAAATTATATGT | |
| | CCGTCGTGTCTTCATCATGGACAACTGCGAAGA | |
| | CCTCATGCCAGAATACTTGAACTTCATCAAGGG | |
| | TGTTGTTGACAGTGAGGATTTGCCGTTGAACAT | |
| | CTCCCGTGAAATGCTCCAACAAAACAAGATCTT | |
| | GAAAGTTATCAGGAAGAATTTGGTTAAGAAAT | |
| | GTTTGGAATTGTTCGAGGAATTGGCTGAAGACA | |
| | AGGACAACTACAAGAAATTGTACGAACAGTTC | |
| | AGCAAGAACTTGAAACTTGGAATCCACGAAGA | |
| | TAGCCAAAACAGAAAGAAACTCTCAGACTTGT | |
| | TGAGATTCCACTCCTCAGCCAGTGGTGACGAAT | |
| | CATGCTCCCTTAAGGAGTATGTTGCACGTATGA | |
| | AGCCAAATCAAACCCACATTTACTACATCACAG | |
| | GTGAAAGCCGTGAACAAGTATCCAACTCTTCAT | |
| | TCGTTGAACGTGTCAAGAAACGTGGTTTTGAAG | |
| | TTATTTACATGACTGAACCCATTGATGAATACG | |
| | TTGTCCAACAAATGAAAGAATATGACGGCAAG | |
| | AACTTGGTATCTGTCACTAAAGAAGGTTTGGAC | |
| | TTGCCTGAAACCGATGAAGAAAAGAAGAAGCG | |
| | CGAGGATGATCAATCCAGATTTGAAAAATTGTG | |
| | CAAAGTTGTTAAGGACATTTTGGACAAGAAAG | |
| | TTGAGAAGGTTGTCATCAGTAACAGACTTGTTG | |
| | AGTCTCCCTGTTGCATTGTCACATCTCAGTATG | |
| | GTTGGACTGCCAACATGGAACGTATCATGAAG | |
| | GCACAAGCACTCAGAGATTCATCTACCATGGGT | |
| | TATATGTCTGCCAAAAAACACTTGGAAATCAAC | |
| | CCTGACCACCCGATCATTGAAACACTCAGACAA | |
| | AAGGCTGAAGCTGATTGCAACGACAAGGCTGT | |
| | CAGAGACTTGGTCATGCTTTTGTTCGAGACAAG | |
| | TTTGTTGTCATCTGGTTTTGGACTTGAAGACCC | |
| | ACAAGTTCACGCTTCTAGAATCCACAGAATGAT | |
| | CAAATTGGGTTTGGGCATTGATGAAGATTTGCC | |
| | AGTAGTTGAAGAAAAATCTGCTGAAGTTGAAG | |
| | CCTCCGAGCCTGTTGTTGAAGCTGATGCTGAAG | |
| | ATTCTTCTCGCATGGAAGAAGTTGAT | |
| ds25 | >CL5923.Contig1_Ag_all eukaryotic | 4 |
| | initiation factor 4A-like | |
| | ATGAATGCTAATGAGACGAAAAATGGACCTCC | |
| | TAGTGAAACCAATGACTACTCGGGACCACCTG | |
| | GCATGGACGTCGGTGGAACTATTGAGTCTGACT | |
| | GGAAAGAAGTGGTGGATAACTTTGATGAGATG | |
| | AATTTAAAAGAAGAATTGTTGCGTGGTATTTAT | |
| | GGATATGGTTTTGAAAAGCCATCAGCTATTCAA | |
| | CAACGTGCTATTTTGCCGTGCATCAAGGGACAT | |
| | GATGTCATTGCTCAGGCCCAATCTGGTACTGGC | |
| | AAGACAGCTACTTTTTCCATTTCTATTCTCCAAC | |
| | AAATTGATACAAGTTTGAATGAGTGCCAAGCA | |
| | CTTATTTTGGCACCAACACGTGAATTGGCTCAA | |
| | CAGATTCAAAAGGTGGTCATTGCTTTGGGTGAT | |
| | TTCATGAAAGCTGATTGTCATGCTTGCATTGGC | |
| | GGTACAAACGTTCGTGATGACATGCGTAAGCTG | |
| | GATACTGGATCCCATGTAGTTGTTGGAACTCCT | |
| | GGCCGTGTTTATGACATGATTGCTAGAAAATCC | |
| | CTAAGAACTCAATTTATCAAGATATTTGTGTTG | |
| | GACGAAGCTGATGAAATGTTGTCTCGAGGTTTC | |
| | AAAGATCAAATTAAAGAGGTGTTCAAGTTCCTC | |
| | GAAGAAGATATTCAGGTCATTCTGTTGTCTGCT | |
| | ACAATGCCCGAGGACGTTTTGGATGTGAGCACT | |
| | CATTTCATGCGTAATCCAGTACGCATTCTTGTTC | |
| | AAAAGGAAGAACTGACATTGGAAGGTATCAAA | |
| | CAGTTTTACATCAATGTTACCAAAGAAGAATGG | |
| | AAGTTTGACACTCTATGTGATTTGTACGACACT | |
| | CTTAGTATCACCCAGGCTGTGATCTTCTGTAAC | |
| | ACACGTCGTAAGGTAGAGTGGTTGACTGAAAA | |
| | TATGCGTTTGAAAACATTTACTGTATCAGCTAT | |
| | GCATGGAGAAATGGACCAACGTCAACGTGAGC | |
| | TAATTATGCGTCAATTCCGTTCTGGCTCTAGTC | |
| | GTGTTCTAATTACCACTGATTTGTTGGCTCGAG | |
| | GCATTGATGTACAACAAGTTTCTCTGGTCATCA | |
| | ATTACGATTTGCCGTCCAATCGTGAAAACTATA | |
| | TTCACAGGATTGGACGTTCTGGCCGTTTCGGTC | |
| | GTAAAGGAGTCGCCATTAATTTTATCACCGAAG | |
| | ACGACAAAAGAGCTATGAAGGATATTGAATCA | |
| | TTTTACAACACTCACGTGCTCGAGATGCCACAG | |
| | AATGTGGCCGATTTGCTG | |
| ds27 | >CL6080.Contig1_Ag_all troponin | 5 |
| | T-like isoform 3 | |
| | ATGTCCGACGAAGAAGAAGTGTACACTGATTC | |
| | CGAAGAAGAAACGCAACCGGAGCCTGAAAAAA | |
| | GCAAAGATGGAGATGGAGATCCCGAATTCGTT | |
| | AAGAGGCAAGAATTAAAATCTTCAGCCTTAGA | |
| | CGAACAGCTTAAAGAGTACATCCAAGAATGGC | |
| | GCAAACAGCGGTCAAAGGAAGAAGACGACTTA | |
| | AAGAAGTTGAAGGAAAAACAGGCCAAGCGCAA | |
| | GGTTATGCGAGCGGAAGAAGAGAAGAGAATGG | |
| | CCGAGAGAAAGAAGCAAGAAGAAGAACGCAG | |
| | ACAGAGAGAAGTCGAGGAAAAGAAACAAAAG | |
| | GACATCGAAGAAAAACGTAAACGTCTAGAAGA | |
| | GGCCGAGAAAAAACGGCAAGCTATGATGGCTG | |
| | CTCTTAAGGAACAAACCAATAAATCTAAAGGA | |
| | CCAAATTTCACCATCAGCAAAAAAGAAGGTGC | |
| | GTTGAGTATGACTTCTGCCCAACTTGAACGCAA | |
| | TAAAACCAGAGAACAGATCGAAGAAGAAAAGA | |
| | AAATATCGTTGAGCTTCAGAATCAAACCTTTGA | |
| | ATATTGAAGGATTCTCTGTGCAAAAACTCCAAT | |
| | TCAAAGCTACCGAACTCTGGGACCAGATCATCA | |
| | AGTTGGAAACAGAAAAATACGATTTGGAGGAA | |
| | AGGCAAAAGAGACAAGATTACGACTTGAAAGA | |
| | GTTGAAAGAACGTCAGAAGCAACAACTCCGCC | |
| | ACAAGGCTCTGAAGAAAGGTCTCGACCCCGAA | |
| | GCCCTAACCGGCAAATACCCACCCAAGATCCA | |
| | AGTCGCTTCCAAGTACGAGAGGCGAGTTGACA | |
| | CGAGGTCTTATGATGACAAAAAGAAGCTGTTC | |
| | GAAGGAGGTTATATGGAAACCACTAAAGAATC | |
| | AATGGAAAAACAATGGACAGAAAAAAGTGACC | |
| | AATTCGGTGGCCGCGCTAAAGGACGATTACCG | |
| | AAATGGTTCGGCGAACGTCCGGGCAAGAAGAA | |
| | GGATGACCCAGACACACCCGAAGAGGAAGAGC | |
| | TCAAGAAAAACGAGGAAGACGAAGAACCGTTT | |
| | GGCCTCGACGACGAAGAAGCTGAAGAAGAAGT | |
| | TGAAGAGGAAGAAGAGGAGGAAGAAGAAGAG | |
| | GAAGAGGAGGAGGAAGAGGAAGAAGAGGAAG | |
| | AAGAAGAAGAGGAAGAGGAAGAAGAAGAAGA | |
| | A | |
| ds45 | >CL2125.Contig1_Ag_all Y-box | 6 |
| | protein Ct-p40-like | |
| | ATGGCGGAACAAGTCGGCGAGAGGAGGACGGA | |
| | ACGGCCGCCGCAGAAGCCCGTGGCCCAAAAGC | |
| | CGGTCATATCTGTGAAAGTCACCGGCGTTGTTA | |
| | AATGGTTCAACGTCAAAAGCGGTTATGGTTTTA | |
| | TTAATCGTAATGATACAAAAGAAGATATATTTG | |
| | TACATCAGTCTGCTATTATCAAGAACAACCCTA | |
| | AGAAAATTGTACGCAGTGTCGGTGATGGAGAA | |
| | ACTGTAGAATTTGACGTTGTTGAGGGCGAAAA | |
| | AGGTCACGAAGCAGCAAATGTTACTGGTCCAG | |
| | ATGGAGAAGCTGTTAAAGGATCACCTTATGCA | |
| | GCTGAAAGAAGAAGAAATAACTATCGTCAGTG | |
| | GTTTTATGGACGCCGTCCTAATACCCGTCCAAG | |
| | AAATGGTGGTCAACCTCCAAGAGATGGTAGTC | |
| | CAAGTGGTGACAAGGAAGAAACTGAAAATGAA | |
| | GTAGGAGAACAACCAAGACGTTACCGCCAGCC | |
| | ACGTCAACAGAATTGGTATAATAGCTATCGTGG | |
| | AAATCGAAGAGGTCCACCACCAAATAGAGGAG | |
| | AAGGTGGTGATTACAATGGTGGAGATAATTAT | |
| | GGATATGATAGTTCACCTCCTGGTAGAGGCAGA | |
| | GGTCGTGGGATGGGTGCGCCTAGACGTTTCTTT | |
| | AGACGTGGCAGTGGATTTAGAGGGAGCCGTGG | |
| | AACAGGTGGTCCACCCAGAAGACCATATCAAG | |
| | ATGAAAATCAGGACAATGAATATAATCAAAGT | |
| | GATGAAAATGGAGCAAATAGACCTCGTCCTCG | |
| | CTATCGCCGCCGCAATAATCGTTCTAGAGCGAG | |
| | AAGTGATGGTCCTCCAAGAGCCAATAGCCAAA | |
| | GTGACAATGAATCTAAACAAAAAAACTTTGGA | |
| | GGAGAAGCATTGGAACTGGATGAAAGTAGTCA | |
| | TGCT | |

### Example 2 the Control Effect of Target Gene dsRNA on Aphids

Inoculating a certain number of green peach aphid or soybean aphid on radish seedlings or soybean seedlings, first, recording the number of aphids inoculated on each plant respectively, and dissolving the synthesized dsRNA into 2% Tween-80, the dsRNA concentrations of the 6 target genes were shown in Table 3. Then spraying 1 ml of dsRNA on the plants inoculated with aphids, and counting on the next day as the statistical results of the first day after dsRNA treatment. Then counting every other day for a total of 3 times and recording as the results of the first day, the third day and the fifth day after treatment, using 2% Tween-80 and dsGFP as a control. The statistical results show that, compared with the control spraying only 2% Tween-80, the control effects of the 3 target genes of the green peach aphid and the 3 target genes of the soybean aphid on the two kinds of aphids all have exceeded 80% (FIG. 1, A, B).

TABLE 3
Spraying concentration of target gene dsRNA

| Gene name of | | | |
|---|---|---|---|
| green peach | Concentration | Gene name of | Concentration |
| aphid | (ng/μl) | soybean aphid | (ng/μl) |
| dsGFP | 295 | dsGFP | 265 |
| ds7 | 233 | ds25 | 282 |
| ds9 | 241 | ds27 | 257 |
| ds15 | 279 | ds45 | 242 |

### Example 3 Statistics of the Dropping Rate of Insect Population of Aphids by Target Genes

Aphids are virginopara insects, born as first-instar newborn aphids. The period from the first instar aphid to the time it can give birth is about 5-7 days (affected by environmental temperature). There are obvious alternation of generations in aphids on a plant, that is, insects of different generations and sizes (different instars) exist at the same time. Therefore, when the test plants are inoculated, there will be aphids of various instars (such as 2th-4th, and there may be adults). In this way, after various test treatments, the aphids quickly begin to reproduce and produce the next generation, resulting in the number of aphids on the tested plant being increased after counting before drug spraying. This is a great interference to the judgment of the control effect of aphids insecticides. Therefore, there is a more rigorous or relatively accurate calculation method for the control effect of aphids, that is, the dropping rate of insect population (see the general methods and materials section for the calculation formula).

The statistical results of the present invention show that after spraying the dsRNA of the 3 green peach aphid target genes, the dropping rates of insect population of the green peach aphid population at 1 day, 3 days and 5 days after treatment are shown in Table 4. The dropping rates of insect population have all reached more than 70% on the 5th day after spraying.

After spraying the dsRNA of the 3 soybean aphid target genes, the dropping rates of insect population of soybean aphid population at 1 day, 3 days and 5 days after treatment are shown in Table 5. The dropping rate of insect population on the 5th day after spraying, except for ds45, the dropping rate of insect population of which is 67.61%, the dropping rates of insect population of the other two target genes are all above 70%.

TABLE 4
The dropping rate of insect population after spraying
with dsRNA of target gene of green peach aphid

| Treatment | 1 d | 3 d | 5 d |
|---|---|---|---|
| CK | −23.55 ± 19.99 | −43.45 ± 41.67 | −111.98 ± 80.5 |
| dsGFP | 10.99 ± 33.56 | −1.61 ± 52.77 | −58.05 ± 74.94 |
| ds7 | 27.73 ± 16.59 | 61.37 ± 17.58 | 74.95 ± 9.11 |
| ds9 | 29.18 ± 21.97 | 63.65 ± 11.32 | 70.35 ± 14.69 |
| ds15 | 38.18 ± 22.79 | 62.66 ± 19.69 | 71.17 ± 13.78 |

TABLE 5
the dropping rate of insect population after spraying
with dsRNA of target gene of soybean aphid

| Treatment | 1 d | 3 d | 5 d |
|---|---|---|---|
| CK | 5.03 ± 13.96 | −50.29 ± 29.93 | −135.23 ± 62.03 |
| dsGFP | −0.75 ± 18.49 | −28.57 ± 19.66 | −59.91 ± 29.55 |
| ds25 | 24.02 ± 18.82 | 67.91 ± 22.1 | 73.85 ± 16.11 |
| ds27 | 33.64 ± 23.56 | 69.28 ± 14.36 | 78.82 ± 10.62 |
| ds45 | 24.59 ± 22.59 | 61.6 ± 21.45 | 67.61 ± 25.64 |

### Example 4 Comparison of the Control Effect of Green Peach Aphid Target and Imidacloprid

Experimental Method:

1. Radish seedlings of 12-15 days, inoculated with 100 insects, stabilized for 1 day, sprayed with dsRNA the next day.

2. The concentration of dsRNA used for spraying was 300 ng/μl. The synthesized dsRNA was dissolved in water and sprayed 300 μl per plant.

3. The concentration of imidacloprid was 10,000 times solution (Germany Bayer Emerald 70% imidacloprid 3 g, water dispersible granules), sprayed 300 μl per plant.

4. The experiment method: spray treatment, 4 replicates for each treatment.

The results are shown in Table 6, Table 7, and FIG. 3 and FIG. 4.

TABLE 6
Field test of green peach aphid: the dropping rate of insect population

| | 1 d | 3 d | 5 d |
|---|---|---|---|
| Control | −116.00 ± 15.06 Aa | −297.87 ± 132.86 Aa | −518.78 ± 161.36 Aa |
| Imidacloprid | 37.33 ± 9.65 Bb | 75.83 ± 15.25 Bb | 84.35 ± 9.60 Bb |
| dsGFP | −67.29 ± 40.98 Aa | −241.98 ± 106.06 Aa | −369.79 ± 131.65 Aa |
| ds7 | 15.75 ± 27.55 Cb | 51.54 ± 10.55 Cb | 81.81 ± 9.47 Bb |
| ds9 | 15.85 ± 16.94 Cb | 66.41 ± 11.20 Bb | 72.64 ± 4.51 Cb |
| ds15 | 21.15 ± 15.10 Bb | 57.65 ± 18.66 Bb | 79.32 ± 4.92 Bb |

TABLE 7
Field test of green peach aphid: Control effect

| | 1 d | 3 d | 5 d |
|---|---|---|---|
| dsGFP | 22.85 ± 15.71 Aa | 12.58 ± 12.90 Aa | 22.88 ± 18.49 Aa |
| ds7 | 61.30 ± 11.62 Bb | 86.26 ± 6.91 Bb | 96.91 ± 1.49 Bb |
| ds9 | 60.56 ± 10.51 Bb | 91.25 ± 2.22 Cb | 95.18 ± 2.07 Bb |
| ds15 | 63.67 ± 4.78 Bb | 89.34 ± 3.21 Cb | 96.51 ± 1.12 Bb |
| Imidacloprid | 70.96 ± 4.25 Cc | 93.78 ± 3.14 Cb | 97.37 ± 1.88 Bb |

The results show that the three genes for green peach aphid have shown obvious lethal effects on the third day, and the dropping rate of insect population is over 70% on the fifth day. Compared with imidacloprid, there is no obvious difference in the dropping rate of insect population. However, compared with the control dsGFP, the dropping rate of insect population shows a significant difference (Table 6); at the same time, the statistical analysis of the control effect shows that the control effect of these three target genes has reached more than 90%, and it can show better control effect on the third day (Table 7). Therefore, this result shows that these three target genes have a strong lethal effect on green peach aphid and can be used as target genes to control green peach aphid for pest control.

### Example 5 Detection of Target Gene Expression

In order to prove that the control effect of spraying dsRNA of these target genes on aphids population is due to the inhibition of the expression of target genes, aphids on day 1, 3, and 5 after treatment with 6 target genes dsRNA were collected, and quantitative PCR (q-RT-PCR) was used to detect whether the target gene was suppressed. The test results of the three target genes of green peach aphid are shown in FIG. 2A. Except for the ds9 gene, the target gene is induced to be up-regulated after 1 day of treatment, all genes are significantly down-regulated after 3 and 5 days of treatment, indicating that the death of aphids is closely related to the level of gene expression.

The detection results of the 3 soybean aphid target genes are shown in FIG. 2B. Except for the expression of the target gene after 1 day of ds25 gene treatment is not significantly different from the control, all genes are significantly down-regulated after 3 and 5 days of treatment, indicating that the death of aphids is closely related to the level of gene expression.

### Example 6

Preparation of the Composition

This example provides a composition for efficiently killing aphids. The composition is an aqueous solution and includes components:
1. The dsRNA for the DS7 gene fragment as shown in SEQ ID NO. 1 or 24 the concentration is 100 ng/μl;
2. The dsRNA for the DS9 gene fragment as shown in SEQ ID NO. 2 or 25 the concentration is 100 ng/μl.
3. The dsRNA for the DS15 gene fragment as shown in SEQ ID NO. 3 or 26 the concentration is 100 ng/μl.
4. The the dsRNA for the DS25 gene fragment as shown in SEQ ID NO. 4 or 27 the concentration is 100 ng/μl.
5. The dsRNA for the DS27 gene fragment as shown in SEQ ID NO. 5 or 28 the concentration is 100 ng/μl.
6. The dsRNA for the DS45 gene fragment as shown in SEQ ID NO. 6 or 29 the concentration is 100 ng/μl.

### Comparative Example 1

The method is the same as that of Examples 1 and 2, the difference is that the target gene is DS50 and the primers used are:

primer F:
(SEQ ID NO.: 30)
TAATACGACTCACTATAGGGAGACGTGTCTGAGGCGGTTGCCA
primer R:
(SEQ ID NO.: 31)
TAATACGACTCACTATAGGGAGATGATCTTGGCCCGGAGAGCCGG

The length of the amplified product is 578 bp.

The DS50 gene is a fatty acid synthase-like gene. The sequence is shown in SEQ ID NO. 23, which encodes the FASN gene. Fatty acid synthase is a multi-enzyme protein that catalyzes fatty acid synthesis. It is not a single enzyme, but an entire enzyme system composed of two identical 272 kDa multifunctional polypeptides, in which the substrate is submitted from one functional domain to the next, and its main function is to catalyze the synthesis of palmitate from acetyl-CoA and malonyl-CoA in the presence of NADPH.

The results show that the dsRNA designed for the DS50 gene by the method of the present invention has a very poor control effect on aphids, with a maximum of only about 23%.

The sequence of the DS50 gene fragment is shown in SEQ ID NO: 23:

(SEQ ID NO.: 23)
TTGGAATTGATTCAACATCTAGCTCAAAGAGGAGCCCGCAAATTTGTTTTA
GTGTCGAAATTGAACAACAAACCTCAGTCAGGTTACAAGACGTTGACCTTA
AGACGGTTGAAGAACAAGAACGTTACCGTAGTCCTATCGTTTGCTGACCCA
TCAACAGTGAGAGGCGCTGAAGACGTACTGAGAGAAGCTGTAGCCCTCGGA
ACAGTCTGTGGTATTTACCACATAACCACCGCTCCGGAAACCAAACACTTG
CAATCCCTGAGCGAAAAGGATTTCGCAGAGACGAAAAAAGTCGTGTCTGAG
GCGGTTGCCAATTTGGACACACTGAGCAGGAGATTGATTCCTCAACTTGAA
TCGTTTGTTGTCCTTGCTCCGGCCGTCGCATCAAGAGGAGCTAAAGCCAAG
TCCAACTACGTTTTCGCAAACGCAGATGTTATCAGAGTCGCTGAAGTCCGT
AAAGTTTCGGGCTATCCAACAGTAGTCATAGAATACGGCGCAATCGAAGGT
ATTTCGAATGCGTTCAACAGTCCAAACTTCAAACCAGCGTCGATCGTTTCA
GCGTTGAATGTTCTGGATGAAATTACCAAACAACCACAAAACCCAACAGTC
GTGTCCTTCTCAAAATTCAACGGTCCAATTTATGAAGAAACGGATGCCGCC
ACTCCATTGTTGAAGACAATTGCCAAGATTTTCGGTTACAAGACACTGTCC
CAAATTGAACAGACCTTTAATCTCGCTCAACTCGGCCTGGACACGTTCCTC
GCACCACGCGTTCAAGAAGCCATCAGACAACAAGCCAACGCAGTCATCGAG
GTAGAAGAACTAAGAACACTGACGTTCCCGGCTCTCCGGGCCAAGATCATC
GAATTACTCGCC

All literatures mentioned in the present application are incorporated by reference herein, as though individually incorporated by reference. Additionally, it should be understood that after reading the above teaching, many variations and modifications may be made by the skilled in the art, and these equivalents also fall within the scope as defined by the appended claims.

1. A dsRNA construct, wherein the dsRNA construct is double-stranded, and its positive or negative strand contains a structure as shown in Formula I:
Seq_{forward}-X-Seqᵣₑᵥₑᵣₛₑ Formula I
wherein
Seq_{forward} is a nucleotide sequence of insect nymph and/or adult stage regulation-related gene or fragment;
Seqᵣₑᵥₑᵣₛₑ is a nucleotide sequence that is basically complementary to Seq_{forward};
X is an intervening sequence between the Seq_{forward} and the Seqᵣₑᵥₑᵣₛₑ, and the intervening sequence is not complementary to the Seq_{forward} and the Seqᵣₑᵥₑᵣₛₑ,
wherein the insect nymph and/or adult stage regulation-related gene is selected from the group consisting of DS7 gene, DS9 gene, DS15 gene, DS25 gene, DS27 gene, DS45 gene and a combination thereof.

2. A dsRNA as shown in Formula II, [chemical structure]
wherein
Seq′_{forward} is a RNA sequence or sequence fragment corresponding to a nucleotide sequence of an insect nymph and/or adult stage regulation-related gene or fragment;
Seq′ᵣₑᵥₑᵣₛₑ is a sequence that is basically complementary to the Seq′_{forward};
X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ;
wherein, the insect nymph and/or adult stage regulation-related gene is selected from the group consisting of: DS7 gene, DS9 gene, DS15 gene, DS25 gene, DS27 gene, DS45 gene, and a combination thereof;
∥ represents the hydrogen bond formed between Seq forward and Seqᵣₑᵥₑᵣₛₑ.

3. The dsRNA of claim 1, the insect is a phytophagous insect, preferably a homoptera insect, most preferably Aphis.

4. An expression vector containing the dsRNA construct of claim 1.

5. A host cell that contains an expression vector containing the dsRNA construct of claim 1 or having the DNA sequence corresponding to the dsRNA construct integrated into the chromosome.

6. A composition comprising the dsRNA construct of claim 1, and an acceptable carrier for insect feeding.

7. A method of:
(1) improving the control effect of aphids;
(2) increasing the dropping rate of insect population;
(3) decreasing the expression level of nymph and/or adult stage regulation-related gene;
(4) reducing the initial number of insect population;
(5) reducing plant damage rate; and/or
(6) reducing crop damage degree and improving the quality of crop products, the method comprising administering the dsRNA construct of claim 1.

8. A method for killing insects, comprising the steps of: using an interference molecule that interferes with the expression of an insect nymph and/or adult stage regulation-related gene, or feeding or spraying an insect with a vector, cell, plant tissue or insect prevention and control reagent containing the interference molecule;
preferably, the insect nymph and/or adult stage regulation-related gene is selected from the group consisting of DS7 gene, DS9 gene, DS15 gene, DS25 gene, DS27 gene, DS45 gene, and a combination thereof.

9. A method for preparing the dsRNA of claim 2, comprising the steps:
(i) preparing a construct expressing dsRNA, and the construct is double-stranded, and its positive or negative strand contains a structure as shown in Formula I:
Seq_{forward}-X-Seqᵣₑᵥₑᵣₛₑ Formula I
wherein
Seq_{forward} is a nucleotide sequence of insect nymph and/or adult stage regulation-related gene or fragment;
Seqᵣₑᵥₑᵣₛₑ is a nucleotide sequence that is basically complementary to Seq_{forward};
X is an intervening sequence located between the Seq_{forward} and the Seqᵣₑᵥₑᵣₛₑ, and the intervening sequence is not complementary to the Seq_{forward} and the Seqᵣₑᵥₑᵣₛₑ,
wherein the insect nymph and/or adult stage regulation-related gene is selected from the group consisting of DS7 gene, DS9 gene, DS15 gene, DS25 gene, DS27 gene, DS45 gene and a combination thereof;
(ii) transforming the construct as described in step (i) into a host cell, thereby expressing and forming a dsRNA as shown in Formula II in the host cell, [chemical structure]
wherein
Seq′_{forward} is a RNA sequence or sequence fragment corresponding to the Seq_{forward} sequence;
Seq′ᵣₑᵥₑᵣₛₑ is a sequence that is basically complementary to the Seq′_{forward};
X′ is none; or is an intervening sequence located between Seq′_{forward} and Seq′ᵣₑᵥₑᵣₛₑ, and the intervening sequence is not complementary to Seq′_{forward} and Seq′ᵣₑᵥₑᵣₛₑ,
∥ represents the hydrogen bond formed between Seq_{forward} and Seqᵣₑᵥₑᵣₛₑ.

10. A method for preparing an insect prevention and control reagent comprising the steps of: spraying the dsRNA construct of claim 1 on the surface of the plant, thereby producing the insect prevention and control agent.

11. A method for improving a plant resistance to an insect, comprising:
expressing a recombinant DNA construct in a plant, wherein the recombinant DNA construct comprises DNA encoding RNA, and the RNA has a sequence that is substantially identical or substantially complementary to at least 21 or more consecutive nucleotides of the target gene, wherein the target gene is an insect nymph and/or adult stage regulation-related gene, selected from the group consisting of DS7 gene, DS9 gene, DS15 gene, DS25 gene, DS27 gene, DS45 gene, and a combination thereof.

12. A method for preparing a transgenic plant cell, comprising the steps:
(i) introducing or transfecting a recombinant DNA construct into a plant cell so that the plant cell contains the construct, thereby producing the transgenic plant cell, wherein the recombinant DNA construct contains DNA encoding RNA, the RNA has a sequence that is substantially identical or substantially complementary to at least 21 or more consecutive nucleotides of the target gene, wherein the target gene is an insect nymph and/or adult stage regulation-related gene, selected from the group consisting of DS7 Gene, DS9 gene, DS15 gene, DS25 gene, DS27 gene, DS45 gene, and a combination thereof.

13. A method for preparing a transgenic plant, comprising the steps:
regenerating a transgenic plant cell prepared by the method of claim 12 into a plant body, thereby obtaining the transgenic plant.

14. A composition comprising the dsRNA of claim 2, and an acceptable carrier for insect feeding.

15. A method of:
(1) improving the control effect of aphids;
(2) increasing the dropping rate of insect population;
(3) decreasing the expression level of nymph and/or adult stage regulation-related gene;
(4) reducing the initial number of insect population;
(5) reducing plant damage rate; and/or
(6) reducing crop damage degree and improving the quality of crop products, the method comprising administering the dsRNA construct of claim 2.

16. A method of:
(1) improving the control effect of aphids;
(2) increasing the dropping rate of insect population;
(3) decreasing the expression level of nymph and/or adult stage regulation-related gene;
(4) reducing the initial number of insect population;
(5) reducing plant damage rate; and/or
(6) reducing crop damage degree and improving the quality of crop products, the method comprising administering the host cell of claim 5.

17. A method of:
(1) improving the control effect of aphids;
(2) increasing the dropping rate of insect population;
(3) decreasing the expression level of nymph and/or adult stage regulation-related gene;
(4) reducing the initial number of insect population;
(5) reducing plant damage rate; and/or
(6) reducing crop damage degree and improving the quality of crop products, the method comprising administering the composition of claim 6.

18. A method of:
(1) improving the control effect of aphids;
(2) increasing the dropping rate of insect population;
(3) decreasing the expression level of nymph and/or adult stage regulation-related gene;
(4) reducing the initial number of insect population;
(5) reducing plant damage rate; and/or
(6) reducing crop damage degree and improving the quality of crop products, the method comprising administering the composition of claim 14.

19. A method for preparing an insect prevention and control reagent comprising the steps of: spraying the dsRNA of claim 2 on the surface of the plant, thereby producing the insect prevention and control agent.

20. A method for preparing an insect prevention and control reagent comprising the steps of: spraying the composition of claim 6 on the surface of the plant, thereby producing the insect prevention and control agent.


SEQUENCE LISTING (31 sequences)

SEQ ID NO: 1 - DNA, 1350 bp, Myzus persicae
atgcgtgaatgtatctctgtacacgttggccaagctggtgttcaaatcggtaatgcctgctgggaattgtactgtttggaacatggaattgctccagatggtcaaatgccatctgacaagaccattggaggtggagacgacagcttcaacaccttcttcagcgaaactggctcaggcaaacatgtgccaagagctgtgttcgttgatctcgaaccaactgttgttgatgaggtaagaactggaacataccgccagttgttccaccctgaacaattgatcactggtaaggaagatgccgccaacaactacgcacgtggacactacactatcggaaaagagattgttgatgttgttttggaccgaatcaggaaattggctgatcagtgcactggtcttcaaggtttcctgatcttccactctttcggaggtggtactggatctggtttcacatctttgttgatggaaagactcagcgttgactacggaaagaagagtaaattagaattcgccatctacccagcccctcaagtatccacagctgtagttgagccatacaactccatcttgaccacacatacaactcttgaacacagtgactgtgcattcatggtcgataatgaagccatctatgacatctgccgtcgtaatctcgatattgaacgtccaacttacactaacttgaatcgtcttattggccagattgtttcttcaatcacagcttctctccgtttcgatggtgccctcaatgttgacttgactgaattccagaccaatttggtcccatacccccgtattcatttcccattggtcacctatgcaccagtcatctccgctgaaaaggcttaccatgaacaattgtccgtatcagaaatcactaacgcttgttttgaaccagccaaccaaatggtgaaatgtgatccacgtcatggcaaatacatggcttgttgcatgttgtaccgtggtgatgttgtacccaaagacgtcaacgctgccattgcttccatcaagaccaagagaacaattcagtttgttgactggtgtccaactggtttcaaagttggtatcaactaccaacccccaaccgtggtacccggtggtgacttggctaaggtacaacgtgccgtctgcatgttgtccaacactacagctattgctgaagcttgggctaggttggaccacaagttcgacttgatgtacgccaaacgtgctttcgtccattggtatgttggagaaggtatggaagaaggagaattctctgaagctcgtgaggatttggctgctctagagaaagattacgaagaggttggcatggactccgtcgaaggcgaaggcgaaggtggtgaagaatac

SEQ ID NO: 2 - DNA, 909 bp, Myzus persicae
atggccgaaaccaaagcgccgaaggacccgtatggtttcttgaaggacttcatggccggtggtatctccgctgccgtgtcgaagaccgccgtggctccgatcgagcgcgtcaagcttatcctgcaagtgcaggccgcttccacgcagatcgccgccgaccaacagtacaaaggaattatggactgtttggtgagaatcccaaaagaacaaggatttgccagtttctggagaggtaactttgccaatgtcatcaggtacttcccaacacaagcattgaactttgctttcaaggatgtctacaaacaggtgtttatggacggtgtggataaaaagactcaattctggcggtattttgctggtaacttggcatctggtggtgctgctggagcaacatctttgtgctttgtataccccctcgattacgcacgtacacgattaggagctgatgtcggtaaaggaccagctgaaaggcagttcaaaggtcttggtgattgtttagccaaaaccgtcaagtctgatggtcccattggtttgtaccgtggtttcattgtatcagtacagggtatcatcatctaccgtgctgcatactttggatttttcgacacagctaagggaatgttgccagaccccaagaatactccattcttagtttcatggggtatcgcccaatttgtaacaacattcgctggtattatgtcctatccatttgacacagtcagacgtcgtatgatgatgcaatctggccgtgctgctgaccaacgcatgtacaagagcacattggactgctggggtaaactttacaagaatgaaggtacatctgctttcttcaagggtgcattctccaacgtactcagaggtactggtggtgccttggtgttggtcttctacgacgaactcaaaaacctcatg

SEQ ID NO: 3 - DNA, 2184 bp, Myzus persicae
atgcctgaagacgttaccatgactgcatctgatgatgttgagaccttcgctttccaagctgagatcgctcagcttatgtccctcatcatcaacaccttctactcgaacaaagaaatctttttgcgagaattggtatccaattcttctgatgcattggacaaaattcgttatgagtcattgactgatccatccaaattggaatctggcaaagatttacacattaaaatcatccccaatgcggaagaaaaaactctgaccattattgacactggtatcggtatgaccaaagctgatctagtcaacaacttgggaaccattgctaaatctggtactaaggctttcatggaagctttacaagctggagctgatatttccatgattggtcaatttggtgtgggtttctattccgcctatctggtagctgacaaagtcactgttgtttccaaacacaacgacgatgaacaatatttgtgggaatctgctgccggaggttcattcaccatccgtactgatcctggtgaaccattgggccgtggtaccaaaattgtccttcaaatcaaagaagatcaagctgagttcctccaacaagaaaaaattaccagcatcatcaagaagcactctcaattcattggctacccaatcaaattaatcgttgagaatgaacgtaccaaagaagtcagcgatgatgaagctgaagaagaaaagaaagatgaagttgaaggtgaaactgaagaagacaaaaaacccaaaattgaggatgttggtgaggatgaagacgaagacaaaaaagatgaagacaaagacaaaaagaagaagaagactattaaagaaaagtacttggatgaagaggtcttgaacaagacaaaaccaatctggacacgcaaccctgatgatatcagccaagatgaatatggtgaattctacaaatccttaaccaatgactgggaagatcatttagccgtcaaacatttctctgtggaaggacaacttgaattcagagcattgttattcattcccaagcgtgcgccttatgacatgtttgagaacaagaagaagaagaacaacattaaattatatgtccgtcgtgtcttcatcatggacaactgcgaagacctcatgccagaatacttgaacttcatcaagggtgttgttgacagtgaggatttgccgttgaacatctcccgtgaaatgctccaacaaaacaagatcttgaaagttatcaggaagaatttggttaagaaatgtttggaattgttcgaggaattggctgaagacaaggacaactacaagaaattgtacgaacagttcagcaagaacttgaaacttggaatccacgaagatagccaaaacagaaagaaactctcagacttgttgagattccactcctcagccagtggtgacgaatcatgctcccttaaggagtatgttgcacgtatgaagccaaatcaaacccacatttactacatcacaggtgaaagccgtgaacaagtatccaactcttcattcgttgaacgtgtcaagaaacgtggttttgaagttatttacatgactgaacccattgatgaatacgttgtccaacaaatgaaagaatatgacggcaagaacttggtatctgtcactaaagaaggtttggacttgcctgaaaccgatgaagaaaagaagaagcgcgaggatgatcaatccagatttgaaaaattgtgcaaagttgttaaggacattttggacaagaaagttgagaaggttgtcatcagtaacagacttgttgagtctccctgttgcattgtcacatctcagtatggttggactgccaacatggaacgtatcatgaaggcacaagcactcagagattcatctaccatgggttatatgtctgccaaaaaacacttggaaatcaaccctgaccacccgatcattgaaacactcagacaaaaggctgaagctgattgcaacgacaaggctgtcagagacttggtcatgcttttgttcgagacaagtttgttgtcatctggttttggacttgaagacccacaagttcacgcttctagaatccacagaatgatcaaattgggtttgggcattgatgaagatttgccagtagttgaagaaaaatctgctgaagttgaagcctccgagcctgttgttgaagctgatgctgaagattcttctcgcatggaagaagttgat

SEQ ID NO: 4 - DNA, 1233 bp, Aphis glycines
atgaatgctaatgagacgaaaaatggacctcctagtgaaaccaatgactactcgggaccacctggcatggacgtcggtggaactattgagtctgactggaaagaagtggtggataactttgatgagatgaatttaaaagaagaattgttgcgtggtatttatggatatggttttgaaaagccatcagctattcaacaacgtgctattttgccgtgcatcaagggacatgatgtcattgctcaggcccaatctggtactggcaagacagctactttttccatttctattctccaacaaattgatacaagtttgaatgagtgccaagcacttattttggcaccaacacgtgaattggctcaacagattcaaaaggtggtcattgctttgggtgatttcatgaaagctgattgtcatgcttgcattggcggtacaaacgttcgtgatgacatgcgtaagctggatactggatcccatgtagttgttggaactcctggccgtgtttatgacatgattgctagaaaatccctaagaactcaatttatcaagatatttgtgttggacgaagctgatgaaatgttgtctcgaggtttcaaagatcaaattaaagaggtgttcaagttcctcgaagaagatattcaggtcattctgttgtctgctacaatgcccgaggacgttttggatgtgagcactcatttcatgcgtaatccagtacgcattcttgttcaaaaggaagaactgacattggaaggtatcaaacagttttacatcaatgttaccaaagaagaatggaagtttgacactctatgtgatttgtacgacactcttagtatcacccaggctgtgatcttctgtaacacacgtcgtaaggtagagtggttgactgaaaatatgcgtttgaaaacatttactgtatcagctatgcatggagaaatggaccaacgtcaacgtgagctaattatgcgtcaattccgttctggctctagtcgtgttctaattaccactgatttgttggctcgaggcattgatgtacaacaagtttctctggtcatcaattacgatttgccgtccaatcgtgaaaactatattcacaggattggacgttctggccgtttcggtcgtaaaggagtcgccattaattttatcaccgaagacgacaaaagagctatgaaggatattgaatcattttacaacactcacgtgctcgagatgccacagaatgtggccgatttgctg

SEQ ID NO: 5 - DNA, 1152 bp, Aphis glycines
atgtccgacgaagaagaagtgtacactgattccgaagaagaaacgcaaccggagcctgaaaaaagcaaagatggagatggagatcccgaattcgttaagaggcaagaattaaaatcttcagccttagacgaacagcttaaagagtacatccaagaatggcgcaaacagcggtcaaaggaagaagacgacttaaagaagttgaaggaaaaacaggccaagcgcaaggttatgcgagcggaagaagagaagagaatggccgagagaaagaagcaagaagaagaacgcagacagagagaagtcgaggaaaagaaacaaaaggacatcgaagaaaaacgtaaacgtctagaagaggccgagaaaaaacggcaagctatgatggctgctcttaaggaacaaaccaataaatctaaaggaccaaatttcaccatcagcaaaaaagaaggtgcgttgagtatgacttctgcccaacttgaacgcaataaaaccagagaacagatcgaagaagaaaagaaaatatcgttgagcttcagaatcaaacctttgaatattgaaggattctctgtgcaaaaactccaattcaaagctaccgaactctgggaccagatcatcaagttggaaacagaaaaatacgatttggaggaaaggcaaaagagacaagattacgacttgaaagagttgaaagaacgtcagaagcaacaactccgccacaaggctctgaagaaaggtctcgaccccgaagccctaaccggcaaatacccacccaagatccaagtcgcttccaagtacgagaggcgagttgacacgaggtcttatgatgacaaaaagaagctgttcgaaggaggttatatggaaaccactaaagaatcaatggaaaaacaatggacagaaaaaagtgaccaattcggtggccgcgctaaaggacgattaccgaaatggttcggcgaacgtccgggcaagaagaaggatgacccagacacacccgaagaggaagagctcaagaaaaacgaggaagacgaagaaccgtttggcctcgacgacgaagaagctgaagaagaagttgaagaggaagaagaggaggaagaagaagaggaagaggaggaggaagaggaagaagaggaagaagaagaagaggaagaggaagaagaagaagaa

SEQ ID NO: 6 - DNA, 909 bp, Aphis glycines
atggcggaacaagtcggcgagaggaggacggaacggccgccgcagaagcccgtggcccaaaagccggtcatatctgtgaaagtcaccggcgttgttaaatggttcaacgtcaaaagcggttatggttttattaatcgtaatgatacaaaagaagatatatttgtacatcagtctgctattatcaagaacaaccctaagaaaattgtacgcagtgtcggtgatggagaaactgtagaatttgacgttgttgagggcgaaaaaggtcacgaagcagcaaatgttactggtccagatggagaagctgttaaaggatcaccttatgcagctgaaagaagaagaaataactatcgtcagtggttttatggacgccgtcctaatacccgtccaagaaatggtggtcaacctccaagagatggtagtccaagtggtgacaaggaagaaactgaaaatgaagtaggagaacaaccaagacgttaccgccagccacgtcaacagaattggtataatagctatcgtggaaatcgaagaggtccaccaccaaatagaggagaaggtggtgattacaatggtggagataattatggatatgatagttcacctcctggtagaggcagaggtcgtgggatgggtgcgcctagacgtttctttagacgtggcagtggatttagagggagccgtggaacaggtggtccacccagaagaccatatcaagatgaaaatcaggacaatgaatataatcaaagtgatgaaaatggagcaaatagacctcgtcctcgctatcgccgccgcaataatcgttctagagcgagaagtgatggtcctccaagagccaatagccaaagtgacaatgaatctaaacaaaaaaactttggaggagaagcattggaactggatgaaagtagtcatgct

SEQ ID NO: 7 - DNA, 39 bp, Artificial Sequence
  Synthetic
taatacgactcactatagggagagacgacggcaactaca

SEQ ID NO: 8 - DNA, 39 bp, Artificial Sequence
  Synthetic
taatacgactcactatagggagaactccagcaggaccat

SEQ ID NO: 9 - DNA, 43 bp, Artificial Sequence
  Synthetic
taatacgactcactatagggagatcgccatctacccagcccct

SEQ ID NO: 10 - DNA, 43 bp, Artificial Sequence
  Synthetic
taatacgactcactatagggagacgggtaccacggttgggggt

SEQ ID NO: 11 - DNA, 43 bp, Artificial Sequence
  Synthetic
taatacgactcactatagggagagccggtggtatctccgctgc

SEQ ID NO: 12 - DNA, 44 bp, Artificial Sequence
  Synthetic
taatacgactcactatagggagatggggtctggcaacattccct

SEQ ID NO: 13 - DNA, 88 bp, Artificial Sequence
  Synthetic
taatacgactcactatagggagatggtgaaccattgggccgtggtaatacgactcactatagggagatggtgaaccattgggccgtgg

SEQ ID NO: 14 - DNA, 43 bp, Artificial Sequence
  Synthetic
taatacgactcactatagggagaaggcgcacgcttgggaatga

SEQ ID NO: 15 - DNA, 43 bp, Artificial Sequence
  Synthetic
taatacgactcactatagggagaccacctggcatggacgtcgg

SEQ ID NO: 16 - DNA, 44 bp, Artificial Sequence
  Synthetic
taatacgactcactatagggagaacgtcctcgggcattgtagca

SEQ ID NO: 17 - DNA, 43 bp, Artificial Sequence
  Synthetic
taatacgactcactatagggagagcgcaaggttatgcgagcgg

SEQ ID NO: 18 - DNA, 43 bp, Artificial Sequence
  Synthetic
taatacgactcactatagggagacggttagggcttcggggtcg

SEQ ID NO: 19 - DNA, 43 bp, Artificial Sequence
  Synthetic
taatacgactcactatagggagagcagaagcccgtggcccaaa

SEQ ID NO: 20 - DNA, 43 bp, Artificial Sequence
  Synthetic
taatacgactcactatagggagataggcgcacccatcccacga

SEQ ID NO: 21 - DNA, 43 bp, Artificial Sequence
  Synthetic
taatacgactcactatagggagacgtgtctgaggcggttgcca

SEQ ID NO: 22 - DNA, 45 bp, Artificial Sequence
  Synthetic
taatacgactcactatagggagatgatcttggcccggagagccgg

SEQ ID NO: 23 - DNA, 879 bp, Aphis glycines
ttggaattgattcaacatctagctcaaagaggagcccgcaaatttgttttagtgtcgaaattgaacaacaaacctcagtcaggttacaagacgttgaccttaagacggttgaagaacaagaacgttaccgtagtcctatcgtttgctgacccatcaacagtgagaggcgctgaagacgtactgagagaagctgtagccctcggaacagtctgtggtatttaccacataaccaccgctccggaaaccaaacacttgcaatccctgagcgaaaaggatttcgcagagacgaaaaaagtcgtgtctgaggcggttgccaatttggacacactgagcaggagattgattcctcaacttgaatcgtttgttgtccttgctccggccgtcgcatcaagaggagctaaagccaagtccaactacgttttcgcaaacgcagatgttatcagagtcgctgaagtccgtaaagtttcgggctatccaacagtagtcatagaatacggcgcaatcgaaggtatttcgaatgcgttcaacagtccaaacttcaaaccagcgtcgatcgtttcagcgttgaatgttctggatgaaattaccaaacaaccacaaaacccaacagtcgtgtccttctcaaaattcaacggtccaatttatgaagaaacggatgccgccactccattgttgaagacaattgccaagattttcggttacaagacactgtcccaaattgaacagacctttaatctcgctcaactcggcctggacacgttcctcgcaccacgcgttcaagaagccatcagacaacaagccaacgcagtcatcgaggtagaagaactaagaacactgacgttcccggctctccgggccaagatcatcgaattactcgcc

SEQ ID NO: 24 - DNA, 634 bp, Myzus persicae
taatacgactcactatagggagatcgccatctacccagcccctcaagtatccacagctgtagttgagccatacaactccatcttgaccacacatacaactcttgaacacagtgactgtgcattcatggtcgataatgaagccatctatgacatctgccgtcgtaatctcgatattgaacgtccaacttacactaacttgaatcgtcttattggccagattgtttcttcaatcacagcttctctccgtttcgatggtgccctcaatgttgacttgactgaattccagaccaatttggtcccatacccccgtattcatttcccattggtcacctatgcaccagtcatctccgctgaaaaggcttaccatgaacaattgtccgtatcagaaatcactaacgcttgttttgaaccagccaaccaaatggtgaaatgtgatccacgtcatggcaaatacatggcttgttgcatgttgtaccgtggtgatgttgtacccaaagacgtcaacgctgccattgcttccatcaagaccaagagaacaattcagtttgttgactggtgtccaactggtttcaaagttggtatcaactaccaacccccaaccgtggtacccgagagggatatcactcagcataat

SEQ ID NO: 25 - DNA, 623 bp, Myzus persicae
taatacgactcactatagggagagccggtggtatctccgctgccgtgtcgaagaccgccgtggctccgatcgagcgcgtcaagcttatcctgcaagtgcaggccgcttccacgcagatcgccgccgaccaacagtacaaaggaattatggactgtttggtgagaatcccaaaagaacaaggatttgccagtttctggagaggtaactttgccaatgtcatcaggtacttcccaacacaagcattgaactttgctttcaaggatgtctacaaacaggtgtttatggacggtgtggataaaaagactcaattctggcggtattttgctggtaacttggcatctggtggtgctgctggagcaacatctttgtgctttgtataccccctcgattacgcacgtacacgattaggagctgatgtcggtaaaggaccagctgaaaggcagttcaaaggtcttggtgattgtttagccaaaaccgtcaagtctgatggtcccattggtttgtaccgtggtttcattgtatcagtacagggtatcatcatctaccgtgctgcatactttggatttttcgacacagctaagggaatgttgccagaccccaagagggatatcactcagcataat

SEQ ID NO: 26 - DNA, 560 bp, Myzus persicae
taatacgactcactatagggagatggtgaaccattgggccgtggtaccaaaattgtccttcaaatcaaagaagatcaagctgagttcctccaacaagaaaaaattaccagcatcatcaagaagcactctcaattcattggctacccaatcaaattaatcgttgagaatgaacgtaccaaagaagtcagcgatgatgaagctgaagaagaaaagaaagatgaagttgaaggtgaaactgaagaagacaaaaaacccaaaattgaggatgttggtgaggatgaagacgaagacaaaaaagatgaagacaaagacaaaaagaagaagaagactattaaagaaaagtacttggatgaagaggtcttgaacaagacaaaaccaatctggacacgcaaccctgatgatatcagccaagatgaatatggtgaattctacaaatccttaaccaatgactgggaagatcatttagccgtcaaacatttctctgtggaaggacaacttgaattcagagcattgttattcattcccaagcgtgcgcctagagggatatcactcagcataat

SEQ ID NO: 27 - DNA, 663 bp, Aphis glycines
taatacgactcactatagggagaccacctggcatggacgtcggtggaactattgagtctgactggaaagaagtggtggataactttgatgagatgaatttaaaagaagaattgttgcgtggtatttatggatatggttttgaaaagccatcagctattcaacaacgtgctattttgccgtgcatcaagggacatgatgtcattgctcaggcccaatctggtactggcaagacagctactttttccatttctattctccaacaaattgatacaagtttgaatgagtgccaagcacttattttggcaccaacacgtgaattggctcaacagattcaaaaggtggtcattgctttgggtgatttcatgaaagctgattgtcatgcttgcattggcggtacaaacgttcgtgatgacatgcgtaagctggatactggatcccatgtagttgttggaactcctggccgtgtttatgacatgattgctagaaaatccctaagaactcaatttatcaagatatttgtgttggacgaagctgatgaaatgttgtctcgaggtttcaaagatcaaattaaagaggtgttcaagttcctcgaagaagatattcaggtcattctgttgtctgctacaatgcccgaggacgtagagggatatcactcagcataat

SEQ ID NO: 28 - DNA, 576 bp, Aphis glycines
taatacgactcactatagggagagcgcaaggttatgcgagcggaagaagagaagagaatggccgagagaaagaagcaagaagaagaacgcagacagagagaagtcgaggaaaagaaacaaaaggacatcgaagaaaaacgtaaacgtctagaagaggccgagaaaaaacggcaagctatgatggctgctcttaaggaacaaaccaataaatctaaaggaccaaatttcaccatcagcaaaaaagaaggtgcgttgagtatgacttctgcccaacttgaacgcaataaaaccagagaacagatcgaagaagaaaagaaaatatcgttgagcttcagaatcaaacctttgaatattgaaggattctctgtgcaaaaactccaattcaaagctaccgaactctgggaccagatcatcaagttggaaacagaaaaatacgatttggaggaaaggcaaaagagacaagattacgacttgaaagagttgaaagaacgtcagaagcaacaactccgccacaaggctctgaagaaaggtctcgaccccgaagccctaaccgagagggatatcactcagcataat

SEQ ID NO: 29 - DNA, 642 bp, Aphis glycines
taatacgactcactatagggagagcagaagcccgtggcccaaaagccggtcatatctgtgaaagtcaccggcgttgttaaatggttcaacgtcaaaagcggttatggttttattaatcgtaatgatacaaaagaagatatatttgtacatcagtctgctattatcaagaacaaccctaagaaaattgtacgcagtgtcggtgatggagaaactgtagaatttgacgttgttgagggcgaaaaaggtcacgaagcagcaaatgttactggtccagatggagaagctgttaaaggatcaccttatgcagctgaaagaagaagaaataactatcgtcagtggttttatggacgccgtcctaatacccgtccaagaaatggtggtcaacctccaagagatggtagtccaagtggtgacaaggaagaaactgaaaatgaagtaggagaacaaccaagacgttaccgccagccacgtcaacagaattggtataatagctatcgtggaaatcgaagaggtccaccaccaaatagaggagaaggtggtgattacaatggtggagataattatggatatgatagttcacctcctggtagaggcagaggtcgtgggatgggtgcgcctaagagggatatcactcagcataat

SEQ ID NO: 30 - DNA, 43 bp, Artificial Sequence; identical to SEQ ID NO: 21
  Synthetic

SEQ ID NO: 31 - DNA, 45 bp, Artificial Sequence; identical to SEQ ID NO: 22
  Synthetic